(19) United States
(12) Patent Application Publication
(10) Pub. No.: US 2006/0183802 A1
(43) Pub. Date: Aug. 17, 2006

(54) Compositions and methods for treating amyotrophic lateral sclerosis (ALS)

(76) Inventors: Wilfredo Colon, Cohoes, NY (US); Jinhyuk Chung, Taejon-City (KR); Mitchel D. De Beus, Cambridge (CA); Curt Breneman, Troy, NY (US); Hongmei Zhang, Randolph, NJ (US)

Correspondence Address: ELMORE PATENT LAW GROUP, PC, 209 MAIN STREET, N. CHELMSFORD, MA, 01863, US

(21) Appl. No.: 11/268,292

(22) Filed: Nov. 4, 2005

Related U.S. Application Data
(63) Continuation of application No. 11/054,695, filed on Feb. 9, 2005, now abandoned.
(60) Provisional application No. 60/543,786, filed on Feb. 11, 2004.

(51) Int. Cl. A61K 31/195; G06F 19/00

(52) U.S. Cl. 514/565; 702/19

(57) ABSTRACT

The present invention provides compositions and methods for treating ALS and other diseases, particularly motor neuron diseases that are mediated by aberrant aggregation of SOD. Patients with ALS may be treated using a compound of the invention which inhibits SOD aggregation mediated by a Cys-111 residue of SOD, or inhibits SOD aggregation mediated by labile SOD beta-barrel ends. The invention also provides methods for designing compounds capable of inhibiting aggregation.

### RELATED APPLICATIONS

This application is a continuation of U.S. application Ser. No. 11/054,695, filed Feb. 9, 2005, which claims the benefit of U.S. Provisional Application No. 60/543,786, filed on Feb. 11, 2004. The entire teachings of the above applications are incorporated herein by reference.

### GOVERNMENT SUPPORT

The invention was supported, in whole or in part, by a grant number R01NS42915 from National Institutes of Health. The Government has certain rights in the invention.

### BACKGROUND OF THE INVENTION

Amyotrophic lateral sclerosis (ALS), also called Lou Gehrig's disease, is a progressive, fatal neurological disease affecting as many as 30,000 Americans with 5,000 new cases occurring in the United States each year. The disorder belongs to a class of disorders known as motor neuron diseases. ALS occurs when specific nerve cells in the brain and spinal cord that control voluntary movement gradually degenerate. Familial amyotrophic lateral sclerosis (FALS) is a form of ALS distinguished from the more common sporadic variant only by its familial background.

Mutations in copper/zinc superoxide dismutase (SOD) are associated with 20% of all cases of familial amyotrophic lateral sclerosis (FALS) (Rosen et al., Nature, 362, (1993) 59-62) and over 100 missense mutations have been identified to date (Andersen et al., ALS and other neuron disorders, 4 (2003)62-67). It has been well established that pathogenic SOD mutations do not cause FALS by interfering with its normal function as a scavenger of the superoxide radical, but rather by acquiring one or more toxic properties (Cleveland et al., Nat. Rev. Neurosci., 2 (2001) 806-819). There have been several hypothesis proposed to explain the toxicity of mutant SOD, including that SOD mutations cause FALS via oxidative damage mediated by SOD bound copper molecule (Liu et al., Biochemistry, 39 (2000) 8125-8132), or alternatively, by inducing the formation of toxic SOD aggregates (Cleveland et al, supra). However, the mechanism by which SOD mutations result in motor neuron death is still under investigation.

Most neurodegenerative diseases have been pathologically linked to the abnormal deposition of a specific protein into fibrillar inclusions known as amyloid (Kaytor et al., J. Biol. Chem., 274 (1999) 37507-37510). It was generally believed that amyloid fibrils were toxic and therefore, the cause of cell death in amyloid diseases. However, evidence has been accumulating in recent years pointing towards soluble oligomers as the toxic species (Bucciantini et al., Nature, 416 (2002) 507-511 and Caughey et al., Annu. Rev. Neurosci., 26 (2003) 267-298). One particular type of oligomer consisting of annular pore-like structure has recently been proposed to be the toxic intermediate in Alzheimer's and Parkinson's diseases (Lashuel et al., Nature, 418 (2002)291). This is consistent with the observation that many proteins linked to various amyloid diseases possess the ability to permeabilize membranes via the formation of ion channels (Kagan et al., Peptides, 23 (2002) 1311-1315).

Recent studies have shown that, upon oxidative damage caused by copper-induced oxidation of metal-depleted SOD, wild-type (WT) and several pathogenic mutants of SOD can form insoluble aggregates, as well as, pore-like oligomeric structures (Chung et al., Biochem. Biophys. Res. Com., 312 (2003) 873-876). Therefore, it would be desirable to inhibit the formation of these toxic SOD aggregates as a therapeutic treatment for ALS, FALS and other diseases such as motor neuron diseases that are mediated by aberrant SOD aggregation.

### SUMMARY OF THE INVENTION

The present invention provides compositions and methods for treating ALS and other diseases such as motor neuron diseases that are mediated by aberrant aggregation of SOD. Patients with ALS may be treated using a compound of the invention which inhibits SOD aggregation mediated by a Cys-111 residue of SOD, or inhibits SOD aggregation mediated by a labile SOD beta-barrel end. The invention also provides methods for designing compounds capable of inhibiting aggregation.

### BRIEF DESCRIPTION OF THE DRAWINGS

FIG. 1 is a ribbon diagram of SOD using the crystal structure (Protein Database Code: 1SPD) and shows the Cys-111 amino acid residue in each monomer at the bottom of the diagram and highlighted using ball and stick format.

FIG. 2A is a line graph showing aggregation of SOD (wild type and A4V, G85R and G37R mutants) in the absence of Cys-111 modification.

FIG. 2B is a line graph showing aggregation of SOD (wild type and A4V, G85R and G37R mutants) after modification of Cys-111 by treatment with iodoacetate.

FIG. 2C is a line graph showing aggregation of SOD (wild type and A4V, G85R and G37R mutants) after modification of Cys-111 by treatment with dinitrobenzoic acid (DTNB).

FIG. 2D is a bar graph showing the extent of final aggregation of SOD (wild type and A4V, G85R and G37R mutants) in the absence of Cys-111 modification.

FIG. 2E is a bar graph showing the extent of final aggregation of SOD (wild type and A4V, G85R and G37R mutants) after modification of Cys-111 by treatment with iodoacetate.

FIG. 2F is a bar graph showing the extent of final aggregation of SOD (wild type and A4V, G85R and G37R mutants) after modification of Cys-111 by treatment with dinitrobenzoic acid (DTNB.)

FIG. 3A is a molecular model of the S5-S6 cleft as presented in spacefill mode. The darkest region is the cleft.

FIG. 3B is a molecular model of the identified binding sites on the S5-S6 cleft. The darkest region consists of residues 74-82 and 102-103. The lower region with less contrast consists of residues 86-88 and 95-99.

FIG. 4A is a molecular model of the S1-S8 cleft in a monomeric SOD. Cys111 is noted as a spatial reference (darkest region on right). The light gray region corresponds to S1-S8 cleft consisting of amino acids 7-11 and 146-147. The darker gray region reflects an auxiliary region consisting of amino acids 53-57.

FIG. 4B is a molecular model of the S1-S8 cleft in a dimeric SOD. The lighter gray region is the S1-S8 cleft. The dark region consists of amino acids 53-57. The cavity formed by the regions leads to the bottom of the dimeric interface.

FIG. 5 shows three diagrams of SOD formations. Panel A is a diagram of the native wild-type (wt) SOD dimeric crystal, panel B is a simplified diagram representing the native wild-type SOD conformation wherein the black dots represent Cys 111 for spatial reference, and panel C is a simplified representation of the reported solution confirmation open confirmation wherein the black dots represent Cys 111 for spatial reference.

FIG. 6 is an energy-minimized binding model of orotate to the Cys-111 crevice. Panel A shows a top view of the Cys 111 crevice and panel B shows a side view of the crevice. The critical region is rendered in surface-characterization mode. The ligands (orotate and water) are shown in spacefill.

FIG. 7 is a molecular model showing ligand-binding site interactions. The ligand is shown in ball and stick representation. Interacting residues are shown in spacefill representations. Lines indicate potential interacting residues but to no represent the actual hydrogen bonds in this model. Panel A shows a top view of the Cys-111 crevice, panel B shows a side view of the ligand-chain “A” interactions, panel C shows a top view of ligand-chain “B” interactions and panel D shows a side view of ligand-chain “B” interactions.

FIG. 8 is a simplified diagram depicting a representative interaction between an orotate molecule and the target binding site (top view). The cavity enclosed by the two half-circles represents the target binding site in the critical region. The dots represent the location of Cys 111 residues. The triangle in the middle represents an orotate molecule with asymmetric interaction groups. The acute angle of the triangle represents carboxylic end.

FIG. 9 is a proposed SOD aggregation mechanism scheme. Small dark dots represent Cys 111 for spatial reference. The highlighted interfaces in the “oligomer stage” represent S5-S6 beta edge of SOD. The aggregation process to the right of the dotted line is described herein. The stages of aggregation to the left of the dotted line outline the inventor's speculation on the aggregation process leading to thioflavin-T positive (ThT(+)) fibrils under conditions proposed by DiDonato et al., infra.

### DETAILED DESCRIPTION OF THE INVENTION

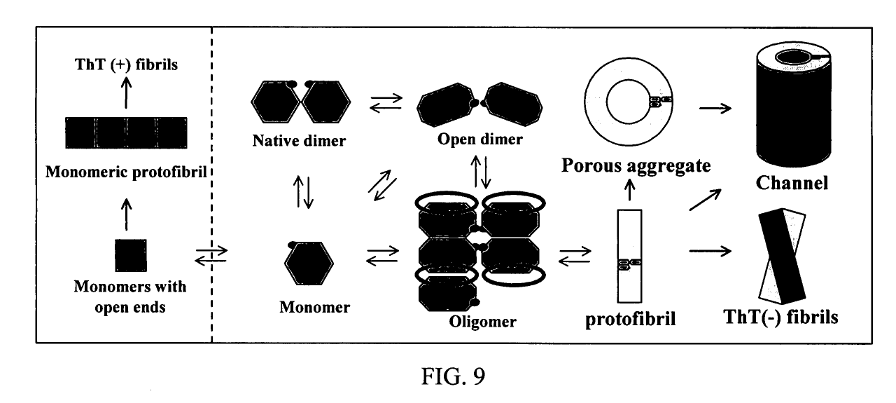

The present inventors have previously discovered that copper-induced oxidation of metal-depleted SOD oxidatively damages SOD and causes its in vitro aggregation into pore-like structures (Chung et al. supra). These pore-like structures are implicated in the pathogenic mechanism of neurodegenerative diseases including ALS, FALS, Parkinson's and Alzheimer's. The inventors have now also discovered that a region of SOD appears to be critical for the copper-induced aggregation of SOD. By blocking this critical region of SOD, particularly the Cys-111 residue, in accordance with the present invention, aggregation of SOD is inhibited. Additionally, the inventors have discovered that in accordance with their SOD aggregation model (see, Example 2 and FIG. 9), the ends of the SOD beta-barrel form a potential site for SOD aggregation and therefore, ligands designed against the barrel ends in accordance with the invention may have synergistic effects in preventing the aggregation of SOD.

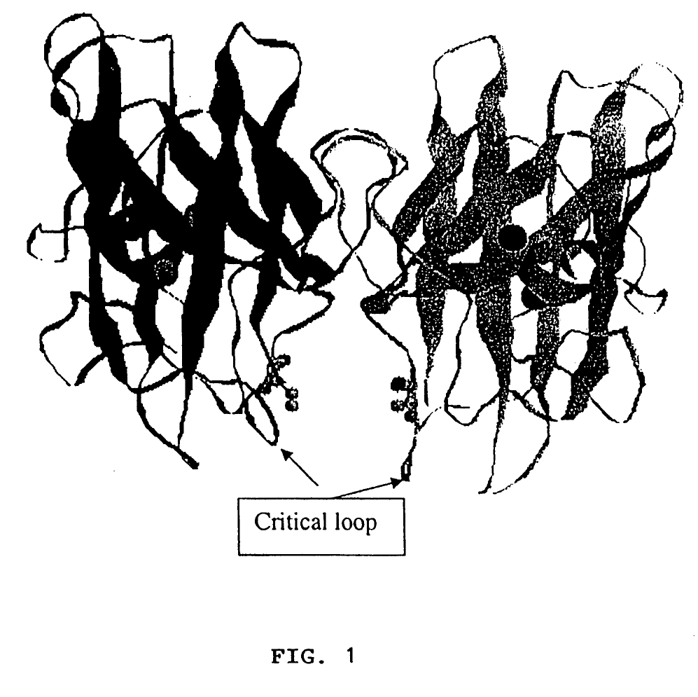

SOD is a 32 kDA homodimer containing one copper ion and one zinc ion in each subunit (see FIG. 1). The copper ion, which alternates between Cu²⁺ and Cu⁺, is the catalytic metal, while the zinc ion Zn²⁺ is believed to play a structural role. Wild type SOD is thermally stable, resistant to denaturants and specific for the superoxide substrate. The amino acid sequence of SOD is shown in SEQ ID NO: 1. From the published human wild type SOD structure (Deng et al. Science 261 (1993) 1047-1051), the Cys-111 is on the surface near the dimer interface and is likely exposed to solvent. Next to Cysteine 111 are histidine 110 and aspartate 109. The distance between the two Cys-111 side chains on each of the dimer units is about 10 Å, as estimated from the crystal structure of the wild type protein (Deng et al. supra).

Additionally, a major structural feature of a SOD subunit is the beta-barrel that forms the core of the structure. The arrangement of beta-sheets in the beta-barrel is exquisite in that it protects the edges of the beta-sheets by connecting them in a circular manner. Such arrangement serves to prevent the aggregation mediated by intermolecular beta-interactions, which can potentially extend indefinitely. The architecture of the SOD beta-barrel, however, is not perfect as it has two “ends” (S5-S6, S1-S8) that may potentially become labile toward intermolecular beta-interactions due to less-than-optimal contacts.

In one aspect, the invention provides methods for designing a pharmaceutically acceptable compound capable of inhibiting aggregation of SOD. The method includes the steps of a) identifying one or more functional groups capable of interacting with one or more regions on the SOD molecule selected from: i) subsites of the critical region of SOD; or ii) subsites of the beta barrel ends of SOD; and (b) identifying a scaffold which presents the functional group or functional groups identified in step (a) in a suitable orientation for interacting with one or more subsites of step (a). The compound which results from attachment of the identified functional groups or moieties to the identified scaffold is a potential inhibitor of SOD aggregation and can be tested for activity. Inhibition of SOD can be tested using a SOD aggregation assay as described herein and an ALS model.

As used herein, the term “SOD aggregation inhibitor” refers to any compound that results in the diminution, inhibition or prevention of SOD aggregation.

As used herein, the term “critical region” of SOD is interchangeable with the term “Cys111 crevice” and both terms refer to that region of SOD that comprises one or more amino acids within 15 angstroms of a Cys-111 residue of SOD as determined from the three dimensional structure of SOD. In one embodiment, the critical region comprises one or more of amino acids 107-115 of SEQ ID NO: 1. In another embodiment the critical region comprises one or more of amino acids 107-113 of SEQ ID NO: 1. In yet another embodiment, the critical region comprises at least amino acids 107-115, or optionally, at least amino acids 107-113 or optionally, at least amino acids 109-111 of SEQ ID NO: 1, and more preferably the critical region comprises at least a Cys-111 residue of SEQ ID NO: 1.

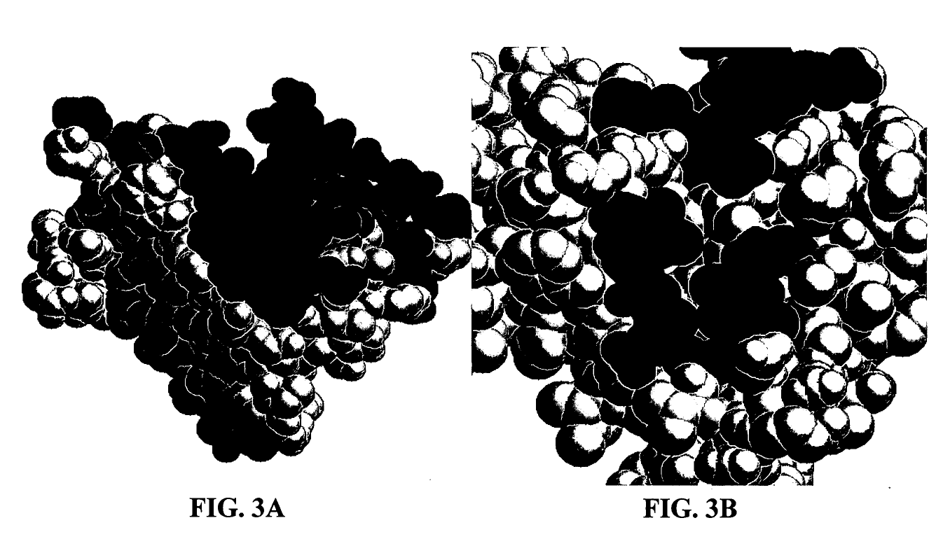

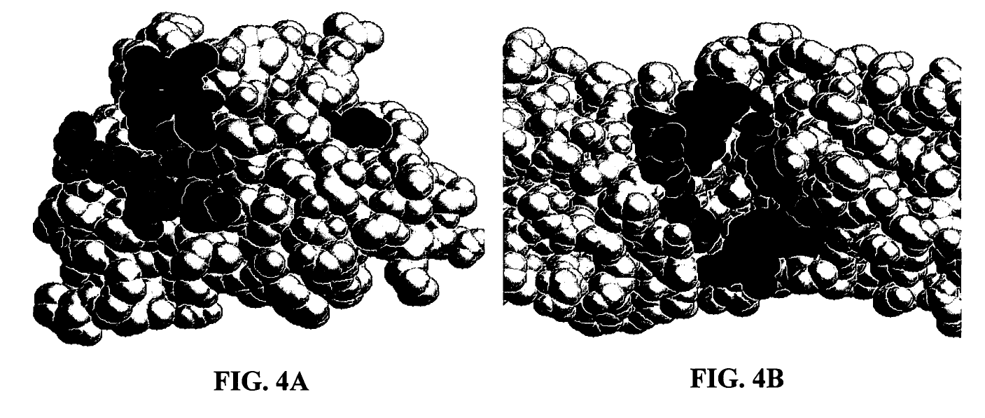

As used herein the “beta barrel ends” of SOD refers to the S5-S6 cleft located at the opposite end of the dimeric interface (FIGS. 3A and 3B) of the native SOD dimer, and the S1-S8 cleft buried within the native SOD dimeric interface (FIGS. 4A and 4B).

As used herein “subsites” are those sites, amino acids, or portions of amino acids located (a) within the critical region, or (b) within the S5-S6 and S1-S8 clefts that make up the beta barrel ends, which may be characterized according to the properties of the chemical moieties to which they are complementary or with which they can interact. Such moieties can include hydrogen bond acceptors (“HA”), such as hydroxyl, amino, and carbonyl groups, halogen atoms, such as fluorine, chlorine, bromine and iodine atoms; and other groups including a heteroatom having at least one lone pair of electrons, such as groups containing trivalent phosphorous, di- and tetravalent sulfur, oxygen and nitrogen atoms; hydrogen bond donors (“HD”), such as hydroxyl, amino, carboxylic acid groups and any of the groups listed under hydrogen atom acceptors to which a hydrogen atom is bonded; hydrophobic groups (“H”), such as linear, branched or cyclic alkyl groups; linear, branched or cyclic alkenyl groups; linear, branched or cyclic alkynyl groups; aryl groups, such as mono- and polycyclic aromatic hydrocarbyl groups and mono- and polycyclic heteroaryl groups; positively charged groups (“P”), such as primary, secondary, tertiary and quaternary ammonium groups, substituted and unsubstituted guanidinium groups, sulfonium groups and phosphonium groups; and negatively charged groups (“N”), such as carboxylate, sulfonamide, sulfamate, boronate, vanadate, sulfonate, sulfinate and phosphonate groups. A given chemical moiety can contain one or more of these groups.

In one embodiment, the invention provides a method of identifying a compound, preferably a pharmaceutically acceptable compound, which inhibits SOD aggregation mediated by a Cys-111 of SOD comprising the steps of: (a) identifying the amino acids within 15 angstroms of Cys-111; (b) rationally designing compounds that will react with one or more amino acids identified in (a); (c) screening the compounds identified in step (b) in a SOD aggregation assay; and optionally, (d) screening the compounds which inhibit SOD aggregation in accordance with step (c) in an amytrophic lateral sclerosis model.

In another embodiment, the invention provides a method of identifying a compound which inhibits SOD aggregation mediated by labile SOD beta barrel ends comprising the steps of: a) identifying the amino acids in and around the S5-S6 cleft and/or the S1-S8 cleft; b) rationally designing compounds that will react with one or more amino acids identified in (a); (c) screening the compounds identified in step (b) in a SOD aggregation assay; and optionally, screening the compounds which inhibit SOD aggregation in accordance with step (c) in an amyotrophic lateral sclerosis model.

Suitable methods, as are known in the art, can be used to identify chemical moieties, fragments or functional groups which are capable of interacting favorably with a particular subsite or set of subsites of (a) the critical region, or (b) the beta barrel ends. These methods include, but are not limited to: interactive molecular graphics; molecular mechanics; conformational analysis; energy evaluation; docking; database searching; pharmacophore modeling; de novo design and property estimation. These methods can also be employed to assemble chemical moieties, fragments or functional groups into a single inhibitor molecule. These same methods can also be used to determine whether a given chemical moiety, fragment or functional group is able to interact favorably with a particular subsite or set of subsites.

A functional group or moiety of the compound is said to “interact” with (a) a subsite of the SOD critical region, or (b) a subsite of the beta barrel ends, if it participates in an energetically favorable, or stabilizing, interaction with one or more complementary moieties within the subsite. Two chemical moieties are “complementary” if they are capable, when suitably positioned, of participating in an attractive, or stabilizing, interaction, such as an electrostatic or van der Waals interaction. Typically, the attractive interaction is an ion-ion (or salt bridge), ion-dipole, dipole-dipole, hydrogen bond, pi-pi or hydrophobic interaction. For example, a negatively charged moiety and a positively charged moiety are complementary because, if suitably positioned, they can form a salt bridge. Likewise, a hydrogen bond donor and a hydrogen bond acceptor are complementary if suitably positioned.

Typically, the assessment of interactions between the test compound and (a) the critical region of SOD, or (b) the beta barrel ends, employs computer-based computational methods, such as those known in the art, in which possible interactions of a compound with the protein, as defined by atomic coordinates, are evaluated with respect to interaction strength by calculating the interaction energy upon binding the compound to the protein. Compounds which have calculated interaction energies within a preselected range or which otherwise, in the opinion of the computational chemist employing the method, have the greatest potential as SOD aggregation inhibitors, can then be provided, for example, from a compound library or via synthesis, and assayed for the ability to inhibit SOD aggregation. The interaction energy for a given compound generally depends upon the ability of the compound to interact with one or more subsites within the SOD critical region and/or the SOD beta barrel ends.

In one embodiment, the design of potential SOD aggregation inhibitors begins from the general perspective of three-dimensional shape and electrostatic complementarity of (a) the critical region, or (b) the beta barrel ends. Subsequently, interactive molecular modeling techniques can be applied by one skilled in the art to visually inspect the quality of the fit of a candidate inhibitor modeled into the critical region or the beta barrel ends. Suitable modeling, simulation and visualization programs include INSIGHTII (Molecular Simulations Inc., San Diego, Calif.), QUANTA (Molecular Simulations Inc., San Diego, Calif.), SYBYL (Tripos Inc., St Louis, Mo.), RASMOL (Roger Sayle et al., Trends Biochem. Sci., 20: 374-376 (1995)), GRASP (Nicholls, et al, Proteins, 11: 281-289 (1991)), MIDAS (Ferrin, et al., J. Mol. Graphics, 6: 13-27(1988), LigBuilder (Wang et al, J. Mol. Model (2000), 6, 498-516), RECON (Sukumar and Breneman (copyright 2003) www.RPI.edu/locker/82/001182/public_html/files/index/html), PEST (Sundling et al., (copyright 2005) www.RPI.edu/locker/82/001182/public_html/files/index/html). A further embodiment of the present invention utilizes a database searching program which is capable of scanning a database of small molecules of known three-dimensional structure for candidates which fit one or more subsites within (a) the critical region, or (b) the beta barrel ends. Suitable software programs include CATALYST (Molecular Simulations Inc., San Diego, Calif.), UNITY (Tripos Inc., St Louis, Mo.), FLEXX (Rarey, et al., J. Mol. Biol., 261: 470-489 (1996)), CHEM-3DBS (Oxford Molecular Group, Oxford, UK), DOCK (Kuntz, et al, J. Mol. Biol., 161:269-288 (1982)), and MACCS-3D (MDL Information Systems Inc., San Leandro, Calif.). It is not expected that the molecules found in the search will necessarily be leads themselves, since a complete evaluation of all interactions will necessarily be made during the initial search. Rather, it is anticipated that such candidates might act as the framework for further design, providing molecular skeletons to which appropriate atomic replacements can be made. Of course, the chemical complimentary of these molecules can be evaluated, but it is expected that the scaffold, functional groups, linkers and/or monomers may be changed to maximize the electrostatic, hydrogen bonding, and hydrophobic interactions with the enzyme. Goodford, J Med Chem, 28:849-857 (1985)) has produced a computer program, GRID, which seeks to determine regions of high affinity for different chemical groups (termed probes) on the molecular surface of the binding site. GRID hence provides a tool for suggesting modifications to known ligands that might enhance binding.

A range of factors, including electrostatic interactions, hydrogen bonding, hydrophobic interactions, desolvation effects, conformational strain, and cooperative motions of ligand and enzyme, all influence the binding effect and should be taken into account in attempts to design bioactive inhibitors.

Yet another embodiment of a computer-assisted molecular design method for identifying inhibitors comprises searching for fragments which fit into a binding region subsite and link to a pre-defined scaffold. The scaffold itself may be identified in such a manner. Programs suitable for the searching of such functional groups and monomers include LUDI (Boehm, J Comp. Aid. Mol. Des., 6:61-78 (1992)), CAVEAT (Bartlett et al., In “Molecular Recognition in Chemical and Biological Problems”, special publication of The Royal Chem. Soc., 78:182-196 (1989)) and MCSS (Miranker, et al. Proteins, 11: 29-34 (1991)).

Yet another embodiment of a computer-assisted molecular design method for identifying SOD aggregation inhibitors of comprises the de novo synthesis of potential inhibitors by algorithmic connection of small molecular fragments that will exhibit the desired structural and electrostatic complementarity with (a) the critical region or (b) the beta barrel ends. The methodology employs a large template set of small molecules which are iteratively pieced together in a model of the SOD critical region. Programs suitable for this task include GROW (Moon, et al., Proteins, 11:314-328 (1991)) and SPROUT (Gillet, et al., J Comp. Aid. Mol. Des., 7:127 (1993)).

In yet another embodiment, the suitability of inhibitor candidates can be determined using an empirical scoring function, which can rank the binding affinities for a set of inhibitors. For an example of such a method see Muegge, et al. and references therein (Muegge, et al., J Med. Chem. 42:791-804 (1999)).

Other modeling techniques can be used in accordance with this invention, for example, those described by Cohen, et al. (J. Med. Chem., 33: 883-894 (1994)); Navia, et al. (Current Opinions in Structural Biology, 2:202-210 (1992)); Baldwin, et al. (J. Med. Chem., 32:2510-2513 (1989)); Appelt, et al. (J. Med. Chem. 34:1925-1934(1991)); and Ealick, et al. (Proc. Nat. Acad. Sci. USA, 88: 11540-11544 (1991)).

A compound which is identified by one of the foregoing methods as a potential inhibitor of SOD can then be obtained, for example, by synthesis or from a compound library, and assessed for the ability to inhibit SOD aggregation in vitro. An in vitro assay for SOD aggregation is described in Chung et al, supra, and in the Examples. Candidate compounds from the in vitro assays can then be screened in any number of ALS models, such as the SOD1-G93 mouse model (Jackson Laboratories, Bar Harbor, Me., see also Journal of Neurochemistry, Vol 71, (1998) 2041-2048) for identifying pharmaceutically acceptable compounds.

Another suitable screening model comprises a high throughput screening assay for determining the inhibitory activity of a compound of the invention against SOD aggregation. This assay is based on the concept that SOD toxicity resulting from SOD aggregation affects mitochondrial respiration by reducing the cytochrome c oxidase (COX) activity, even at a very early age in mice model (Kirkinezos, I. G. et al., J Neurosci 25, 164-72 (2005)). Hence, using the technique of COX activity assay on cells containing intact mitochondria using a microtiter plate reader as proposed by Chrzanowska-Lightowlers et al. (Chrzanowska-Lightowlers et al., Anal Biochem 214, 45-9 (1993)) in neuronal cell lines (i.e. neuroblastoma cells) expressing mutant SOD (i.e. G93A mutant, G37R, A4V, etc) would enable high throughput screening of active compounds.

Briefly, the procedure would consist of preparation of neuronal cells expressing a mutant SOD in 96-well microtiter plates, which would be grown for a fixed length of time in the presence of prospective drug compounds identified in accordance with the present invention. Then, the plates would be assayed for COX activity assay to screen for the compounds that prevented the reduction of COX activity.

Preferred therapeutic compounds designed in accordance with the invention have a molecular weight of less than 2000 g/mol. More preferably, compounds designed in accordance with the invention have a molecular weight of less than 1000 g/mol.

In one preferred embodiment, a pharmaceutically acceptable SOD aggregation inhibitor designed to target the critical Cys-111 region of SOD comprises one or more of the following: (a) a chemical moiety comprising a functional group capable of interacting with the sulfhydryl side chain of a Cys-111 of SOD (b) a chemical moiety positioned to interact with the acidic side-chain of an Asp-109 of SOD; (c) a chemical moiety positioned to interact with the imidazole of His 110 of SOD; (d) a chemical moiety capable of specifically binding Cys-111; (e) a chemical moiety capable of specifically binding Cys-111 and one or more amino acids within 5 amino acid residues of Cys-111; (f) a chemical moiety capable of binding one or more amino acids within 5 amino acid residues of Cys-111; (g) a chemical moiety capable of binding one or more amino acids located within 15 Å of Cys-111; (h) a chemical moiety capable of binding one or more amino acids located within 10 Å of Cys-111; (i) a chemical moiety capable of binding a basic amino acid within 5 amino acid residues of Cys-111; (j) a chemical moiety capable of binding a basic amino acid located within 10 Å of Cys-111; (k) a chemical moiety capable of binding a hydrophobic amino acid within 5 amino acid residues of Cys-111, (1) a chemical moiety capable of binding a hydrophobic amino acid within 10 Å of Cys-111; (m) a chemical moiety capable of binding an acidic amino acid within 5 amino acid residues of Cys-111; (n) a chemical moiety capable of binding an acidic amino acid located within 10 Å of Cys-111.

In one preferred embodiment, a pharmaceutically acceptable SOD inhibitor is capable of blocking an amino acid in the critical Cys-11 region of SOD. In another preferred embodiment, a SOD inhibitor is capable of blocking a Cys-111 amino acid residue of SOD. A compound is said to “block” an amino acid if it interacts with the amino acid and prevents the amino acid from undergoing any further chemical reaction.

In one preferred embodiment, a computational search for a commercially available binding ligand to the critical Cys-111 region initially yielded 4-pyrimidinecarboxylic acid as shown in Formula 1 as a potential binding ligand. The compound of Formula 1 has the following structure: 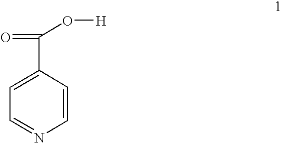
The compound of Formula 1 was structurally similar to orotic acid (vitamin B13) shown in Formula 2, which is well characterized and commercially available. The compound of Formula 2 has the following structure at pH 7: 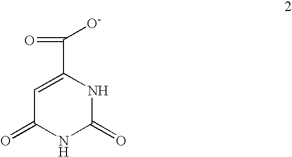
In addition, orotic acid is a biological intermediate for nucleic acid synthesis (23) and it is widely utilized as a counter-ion for mineral intake. At physiological pH, orotic acid is mainly found in carboxylate form with a deprotonated carboxylic acid group (24). Intrigued by the fact that orotic acid is a biological intermediate, we evaluated computational binding score of orotic acid to the critical region and found greatly enhanced binding score in comparison to 4-pyrimidinecarboxylic acid.

The binding model of orotate to the Cys111 crevice was studied in further detail through energy minimization and docking simulations. The energy minimized binding model showed placement of orotate between the Cys111 residues, where the site was characterized by a mixture of hydrophilic and hydrophobic patches (FIG. 6). A docking simulation, which uses similar energy minimization algorithm, produced a list of possible binding modes with different interactions between orotate and Cys111 crevice. The computed binding energy contribution from van der Waals, electrostatic, ligand conformation, and de-solvation, suggested de-solvation as the main driving force for the binding of the ligand. This computation result is in agreement with the fact that orotic acid is poorly soluble in water (solubility in water at 7 mM according to MSDS). Indeed, surface characterization of orotate showed a large hydrophobic surface on each side of the ring, further supporting the computational result.

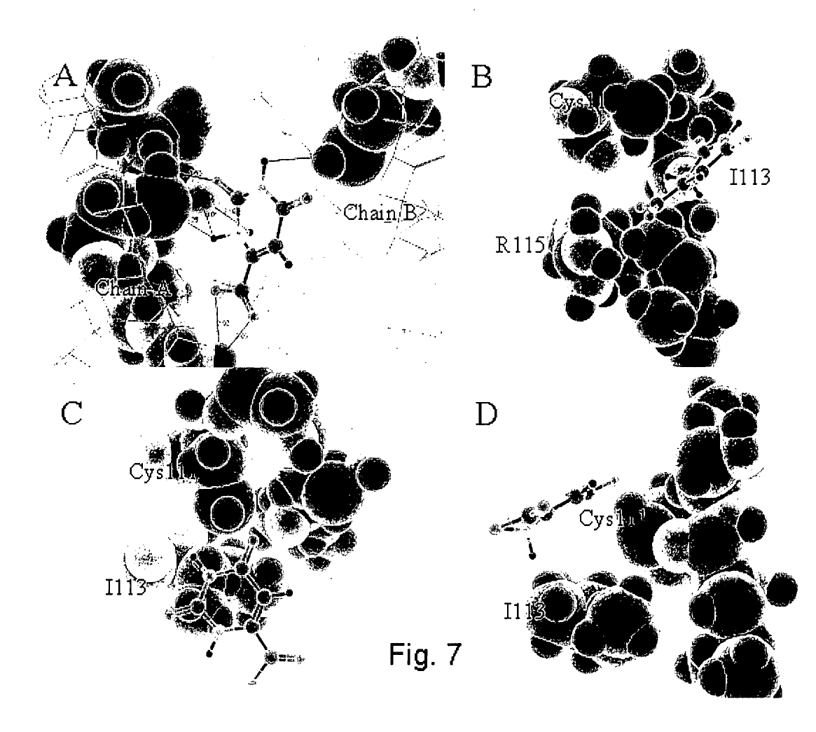

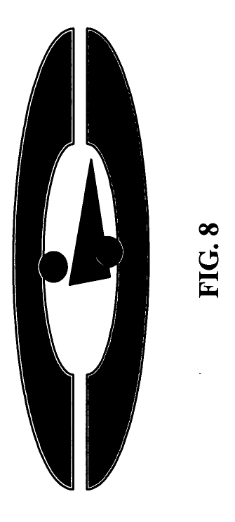

A more detailed analysis on the energy-minimized binding model showed important interaction features between orotate and the Cys111 crevice (FIG. 7) of the critical region. The carboxylate group of orotic acid appeared to be within 3.3 A of positively charged amine group of arginine 115, suggesting a possible salt-bridge formation. Dioxy-pyridine ring, on the other hand, appeared to have numerous potential hydrogen bonding interactions with Cys111 and isoleucine 113 (I113) through the backbone oxygen. Interestingly, the majority of interactions between the SOD dimer and the orotate appeared to be focused on a single subunit (namely, chain A) (FIG. 7, Panel B). Chain B, on the other hand, had only a couple of unfavorable potential hydrogen bonding interactions with the orotate through the Cys111 sulfhydryl or the backbone oxygen of I113 (FIG. 7, Panel C and D). Such asymmetric interaction between orotate and SOD subunits is likely due to the small size and the asymmetry in the orotate H-bonding groups as represented in the simplified diagram (FIG. 8). From the simplified diagram, it is easier to see that the specific interactions between the ligand and the target site may not contribute significantly to the dimeric stability.

In another preferred embodiment, a binding ligand to the critical Cys-111 region in accordance with the invention is a substituted guanidine, such as a compound represented by the Formula 3:

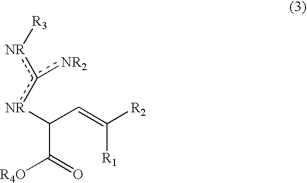

Wherein R₁ is hydrogen, a substituted or unsubstituted aliphatic or aromatic group, preferably a hydrophobic aliphatic group, such as a substituted or unsubstituted, saturated or unsaturated alkyl group. A preferred R₁ group is a C₃-C₉ alkyl, such as a straight or branched chain butyl, pentyl, hexyl or octyl group.

R₂ is a substituted or unsubstituted aliphatic or aromatic group. Preferably R₂ is a substituted or unsubstituted, saturated or unsaturated alkyl group, such as a C₂-C₅ alkyl or alkenyl. Particularly preferred R₂ groups include substituted or unsubstituted ethyl and propenyl.

R₃ is a substituted or unsubstituted aliphatic or aromatic group, preferably a substituted methylene.

R₄ is hydrogen, or an aliphatic or aromatic group.

The dashed bond represents a single bond, a double bond or a tautomer.

Each R at a guanidino nitrogen is independently absent or selected from a hydrogen, a substituted or unsubstituted aliphatic or aromatic group, preferably a hydrogen or methyl. In general, one R is absent and one bond is a double bond. In a most preferred embodiment, each remaining R is a hydrogen.

A preferred structure has the formula 4: 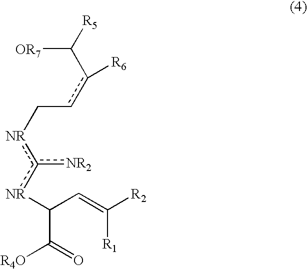
Wherein R, R₁, R₂ and R₄ are defined above;
R₅ is H, carboxylic acid, sulfonic acid, sulfate and esters thereof, amino, amido, imino, hydroxy or an aliphatic or aromatic group. A preferred R₅ group is a substituted or unsubstituted straight or branched chain, saturated or unsaturated C₁-C₈ alkyl, such as ethyl or pentyl;
R₆ is H, carboxy, sulfonic acid, sulfate and esters thereof, amino, amido, urea, acylurea, ureacarbonyl hydroxy, nitroso, nitro, or an aliphatic or aromatic group, such as a substituted or unsubstituted, saturated or unsaturated alkyl;
or R₅ and R₆ can be taken together to form a ring, including substituted or unsubstituted polycyclic ring systems;
R₇ is a hydrogen, substituted or unsubstituted alkyl, acyl group, or protecting group and the dashed bond represents a single bond, a double bond or a tautomer.

Particularly interesting compounds of the invention include dimers of the compounds discussed above. Taking the compounds of formula 3, a preferred dimer occurs at R₃ wherein the R₃ group has the structure L-R₈ where R₈ has the formula 3, bound to L via the R₃ and L is a ligand, such as a C2-C8 substituted or unsubstituted alkylene.

The compounds also include prodrugs, salts, enantiomers, diastereoisomers, racemates, and tautomers of the compounds.

Definitions

Listed below are definitions of various terms used to describe this invention. These definitions apply to the terms as they are used throughout this specification and claims, unless otherwise limited in specific instances, either individually or as part of a larger group.

An “aliphatic group” is non-aromatic moiety that may contain any combination of carbon atoms, hydrogen atoms, halogen atoms, oxygen, nitrogen, sulfur or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

Suitable aliphatic or aromatic substituents include, but are not limited to, —F, —Cl, —Br, —I, —OH, protected hydroxy, aliphatic ethers, aromatic ethers, oxo, —NO₂, —NO, —NR₁₀R₁₁ wherein R₁₀ and R₁₁ are independently selected from H, substituted or unsubstituted, saturated or unsaturated, alkyl and heterocycles (such as azoles), —CN, —C₁-C₁₂-alkyl optionally substituted with, for example, halogen (such as perhaloalkyls), C₂-C₁₂-alkenyl optionally substituted with, for example, halogen, —C₂-C₁₂-alkynyl optionally substituted with, for example halogen, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH -heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkynyl, —CONH—C₃-C₁₂-cycloalkyl, —CONH-aryl, —CONH-heteroaryl, —CONH-heterocycloalkyl, —CO₂—C₁-C₁₂-alkyl, —CO₂—C₂-C₁₂-alkenyl, —CO₂—C₂-C₁₂-alkynyl, —CO₂—C₃-C₁₂-cycloalkyl, —CO₂-aryl, —CO₂-heteroaryl, —CO₂-heterocycloalkyl, —OCO₂—C₁-C₁₂-alkyl, —OCO₂—C₂-C₁₂-alkenyl, —OCO₂—C₂-C₁₂-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, NHC(O)NH—C₁-C₁₂-alkyl, —NHC(O)NH—C₂-C₁₂-alkenyl, —NHC(O)NH—C₂-C₁₂-alkynyl, —NHC(O)NH—C₃-C₁₂-cycloalkyl, —NHC(O)NH-aryl, —NHC(O)NH-heteroaryl, —NHC(O)NH-heterocycloalkyl, —CONHC(O)NH₂, —CONHC(O)NH—C₁-C₁₂-alkyl, —CONHC(O)NH—C₂-C₁₂-alkenyl, —NHC(O)NHCO—C₂-C₁₂-alkynyl, —NHC(O)NHCO—C₃-C₁₂-cycloalkyl, —NHC(O)NHCO-aryl, —NHC(O)NHCO-heteroaryl, —NHC(O)NHCO-heterocycloalkyl, NHC(S)NH₂, NHC(S)NH—C₁-C₁₂-alkyl, —NHC(S)NH—C₂-C₁₂-alkenyl, —NHC(S)NH—C₂-C₁₂-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, NHC(NH)NH—C₁-C₁₂-alkyl, —NHC(NH)NH—C₂-C₁₂-alkenyl, —NHC(NH)NH—C₂-C₁₂-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, NHC(NH)—C₁-C₁₂-alkyl, —NHC(NH)—C₂-C₁₂-alkenyl, —NHC(NH)—C₂-C₁₂-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkynyl, —S(O)—C₃-C₁₂-cycloalkyl, —S(O)-aryl, —S(O)-heteroaryl, —S(O)-heterocycloalkyl—SO₂NH₂, —SO₂NH—C₁-C₁₂-alkyl, —SO₂NH—C₂-C₁₂-alkenyl, —SO₂NH—C₂-C₁₂-alkynyl, —SO₂NH—C₃-C12-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkynyl, —S—C₃-C₁₂-cycloalkyl, —S-aryl, —S-heteroaryl, —S-heterocycloalkyl, or methylthiomethyl. It is understood that the aryls, heteroaryls, alkyls and the like can be further substituted.

The terms “C₁-C₃ alkyl”, “C₁-C₆ alkyl”, “C₁-C₁₂ alkyl”, or “C₃-C₉ alkyl”, as used herein, refer to saturated, straight- or branched-chain hydrocarbon radicals containing between one and three, one and twelve, or one and six carbon atoms, respectively. Examples of C₁-C₃ alkyl radicals include methyl, ethyl, propyl and isopropyl radicals; examples of C₁-C₆ alkyl radicals include, but are not limited to, methyl, ethyl, propyl, isopropyl, n-butyl, tert-butyl, sec-butyl, n-pentyl, neopentyl and n-hexyl radicals; and examples of C₁-C₁₂ alkyl radicals include, but are not limited to, ethyl, propyl, isopropyl, hexyl, heptyl, octyl, nonyl, decyl, undecyl, dodecyl radicals and the like. Examples of C3-C9 alkyl radicals include for example, straight or branched chain pentyl or hexyl radicals and the like.

The term “substituted alkyl,” as used herein, refers to an alkyl, such as a C₁-C₁₂ alkyl or C₁-C₆ alkyl group, substituted by one, two, three or more aliphatic substituents.

The terms “C₂-C₁₂ alkenyl” or “C₂-C₆ alkenyl,” as used herein, denote a monovalent group derived from a hydrocarbon moiety containing from two to twelve or two to six carbon atoms having at least one carbon-carbon double bond by the removal of a single hydrogen atom. Alkenyl groups include, but are not limited to, for example, ethenyl, propenyl, butenyl, 1-methyl-2-buten-1-yl, alkadienes and the like.

The term “substituted alkenyl,” as used herein, refers to a “C₂-C₁₂ alkenyl” or “C₂-C₆ alkenyl” group as previously defined, substituted by one, two, three or more aliphatic substituents.

The terms “C₂-C₁₂ alkynyl” or “C₂-C₆ alkynyl,” as used herein, denote a monovalent group derived from a hydrocarbon moiety containing from two to twelve or two to six carbon atoms having at least one carbon-carbon triple bond by the removal of a single hydrogen atom. Representative alkynyl groups include, but are not limited to, for example, ethynyl, 1-propynyl, 1-butynyl, and the like.

The term “substituted alkynyl,” as used herein, refers to a “C₂-C₁₂ alkynyl” or “C₂-C₆ alkynyl” group as previously defined, substituted by one, two, three or more aliphatic substituents.

The term “C₁-C₆ alkoxy,” as used herein, refers to a C₁-C₆ alkyl group, as previously defined, attached to the parent molecular moiety through an oxygen atom. Examples of C₁-C₆-alkoxy include, but are not limited to, methoxy, ethoxy, propoxy, isopropoxy, n-butoxy, sec-butoxy, tert-butoxy, n-pentoxy, neopentoxy and n-hexoxy.

The terms “halo” and “halogen,” as used herein, refer to an atom selected from fluorine, chlorine, bromine and iodine.

The terms “aryl” or “aromatic” as used herein, refer to a mono- or bicyclic carbocyclic ring system having one or two aromatic rings including, but not limited to, phenyl, naphthyl, tetrahydronaphthyl, indanyl, idenyl and the like.

The terms “substituted aryl” or “substituted aromatic,” as used herein, refer to an aryl or aromatic group substituted by one, two, three or more aromatic substituents.

The term “arylalkyl,” as used herein, refers to an aryl group attached to the parent compound via a C₁-C₃ alkyl or C₁-C₆ alkyl residue. Examples include, but are not limited to, benzyl, phenethyl and the like.

The term “substituted arylalkyl,” as used herein, refers to an arylalkyl group, as previously defined, substituted by one, two, three or more aromatic substituents.

The terms “heteroaryl” or “heteroaromatic,” as used herein, refer to a mono-, or polycyclic (e.g. bi-, or tri-cyclic or more) aromatic radical or ring having from five to ten ring atoms of which at least one or more ring atom is selected from, for example, S, O and N; zero, one, two or more ring atoms are additional heteroatoms independently selected from, for example, S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The heteroaromatic ring may be bonded to the chemical structure through a carbon or hetero atom.

The terms “substituted heteroaryl” or “substituted heteroaromatic,” as used herein, refer to a heteroaryl or heteroaromatic group, substituted by one, two, three, or more aromatic substituents.

The term “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo [2.2.1] heptyl, and bicyclo [2.2.2] octyl.

The term “substituted alicyclic,” as used herein, refers to an alicyclic group substituted by one, two, three or more aliphatic substituents.

The term “heterocyclic,” as used herein, refers to a non-aromatic ring, comprising three or more ring atoms, or a bi- or tri-cyclic group fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl.

The term “substituted heterocyclic,” as used herein, refers to a heterocyclic group, as previously defined, substituted by one, two, three or more aliphatic substituents.

The term “heteroarylalkyl,” as used herein, to an heteroaryl group attached to the parent compound via a C₁-C₃ alkyl or C₁-C₆ alkyl residue. Examples include, but are not limited to, pyridinylmethyl, pyrimidinylethyl and the like.

The term “substituted heteroarylalkyl,” as used herein, refers to a heteroarylalkyl group, as previously defined, substituted by independent replacement of one, two, or three or more aromatic substituents.

The term “alkylamino” refers to a group having the structure —NH(C₁-C₁₂ alkyl).

The term “dialkylamino” refers to a group having the structure —N(C₁-C₁₂alkyl) (C₁-C₁₂alkyl) and cyclic amines. Examples of dialkylamino are, but not limited to, dimethylamino, diethylamino, methylethylamino, piperidino, morpholino and the like.

The term “alkoxycarbonyl” represents an ester group, i.e., an alkoxy group, attached to the parent molecular moiety through a carbonyl group such as methoxycarbonyl, ethoxycarbonyl, and the like.

The term “carboxaldehyde,” as used herein, refers to a group of formula —CHO.

The term “carboxy,” as used herein, refers to a group of formula —COOH.

The term “carboxamide,” as used herein, refers to a group of formula —C(O)NH(C₁-C₁₂alkyl) or —C(O)N(C₁-C₁₂alkyl) (C₁-C₁₂ alkyl), —C(O)NH₂, NHC(O)(C₁-C₁₂alkyl), N(C₁-C₁₂alkyl)C(O)(C₁-C₁₂alkyl) and the like.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl- 3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bz or —C(O)C₆H₅), and trimethylsilyl (TMS or —Si(CH₃)₃).

The term “protected hydroxy,” as used herein, refers to a hydroxy group protected with a hydroxy protecting group, as defined above, including benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bz or —C(O)C₆H₅), and trimethylsilyl (TMS or —Si(CH₃)₃).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “protected amino,” as used herein, refers to an amino group protected with an amino protecting group as defined above.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of the invention. Various forms of prodrugs are known in the art, for example, as discussed in Bundgaard, (ed.), Design of Prodrugs, Elsevier (1985); Widder, et al. (ed.), Methods in Enzymology, vol.4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

As used herein, the term SOD aggregation-mediated disease(s) refers to any disease in which SOD aggregation is implicated in the onset and/or pathological progress of the disease, including certain motor neuron diseases. SOD aggregation-mediated motor neuron disease includes but is not limited to neurodegenerative disorders such as, Parkinson's disease, Huntington's disease, Alzheimer's disease, Hallervorden-Spatz disease, olivopontocerebellar atrophy, multiple system atrophy, progressive supranuclear palsy, diffuse lewy body disease, corticodentatonigral degeneration, progressive familial myoclonic epilepsy, strionigral degeneration, torsion dystonia, familial tremor, gilles de la tourette syndrome, and ALS which is familial, sporadic typical, or atypical in nature.

Preferred binding ligands to the critical Cys-111 region in accordance with the invention include compounds having the following formulas:

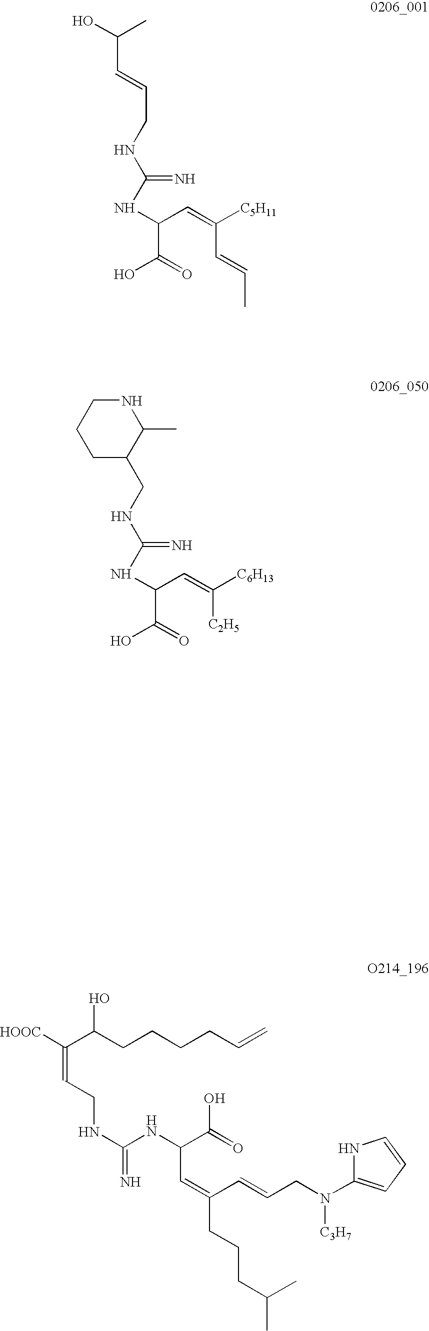

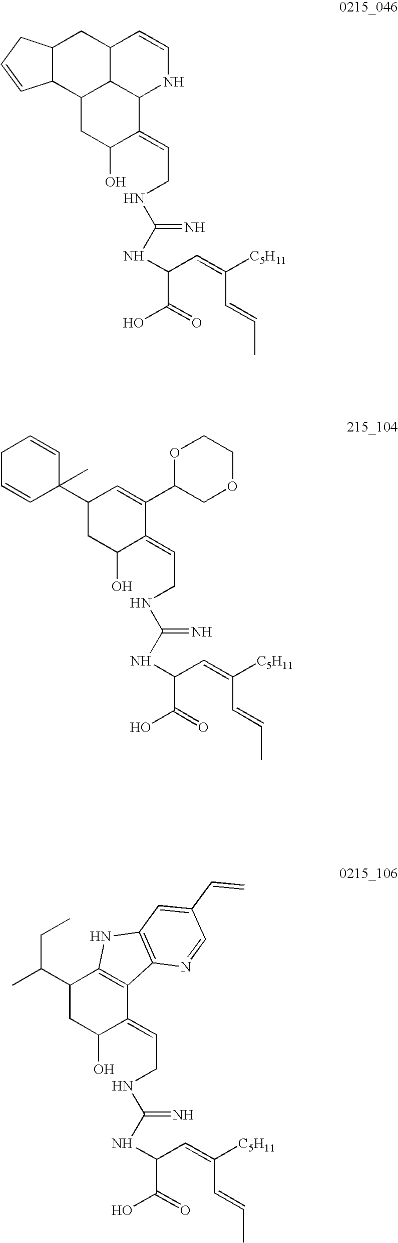

Other compounds that can be used in the invention include:

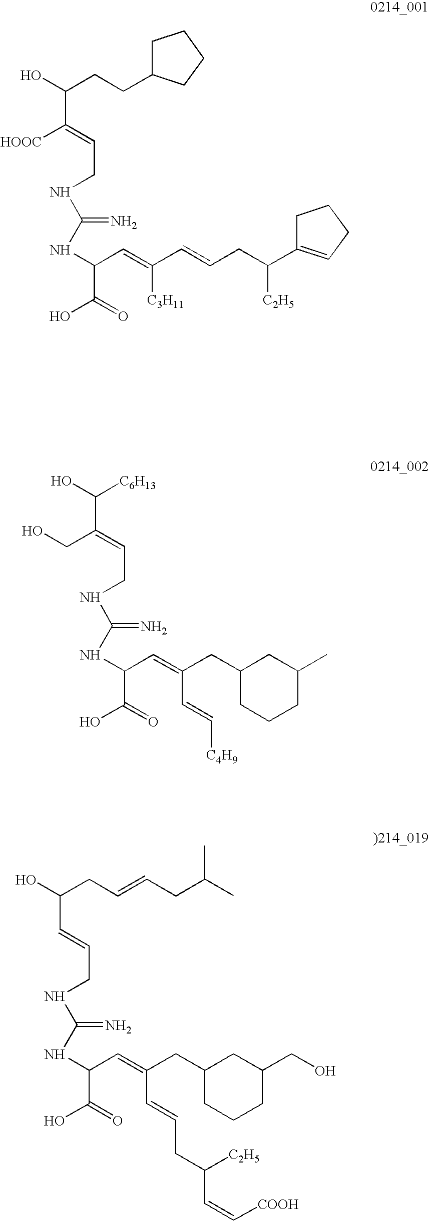

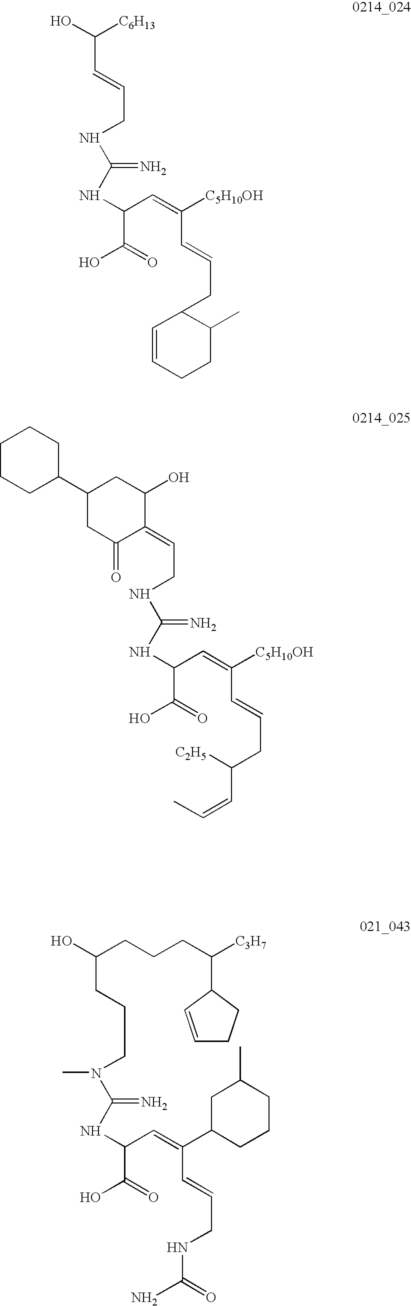

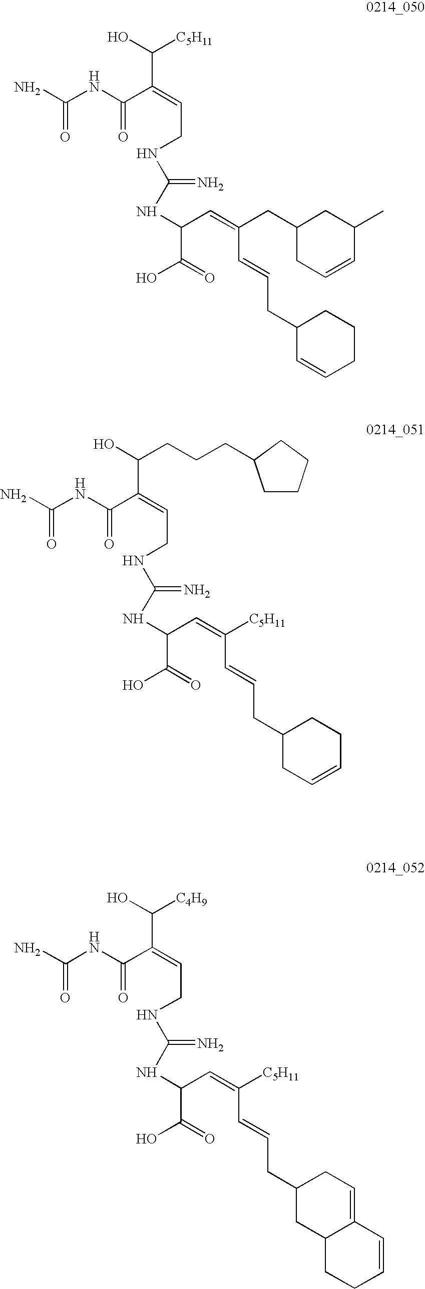

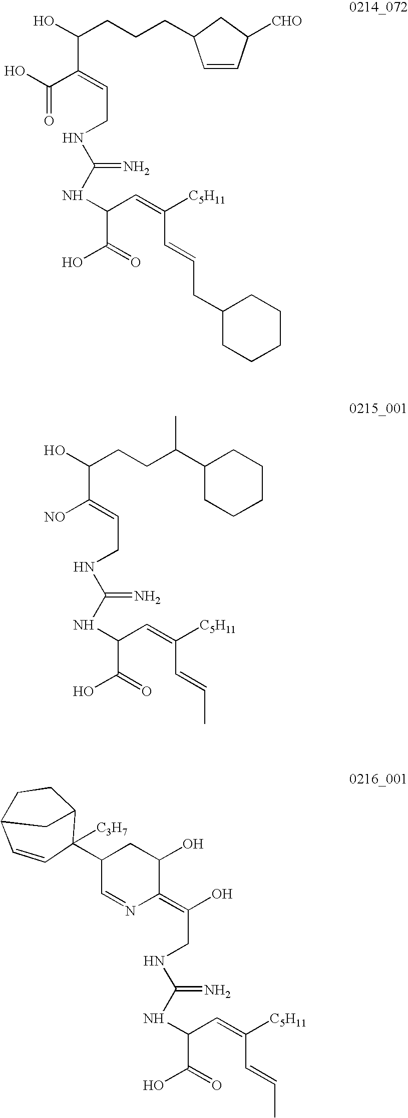

Yet other compounds may include:

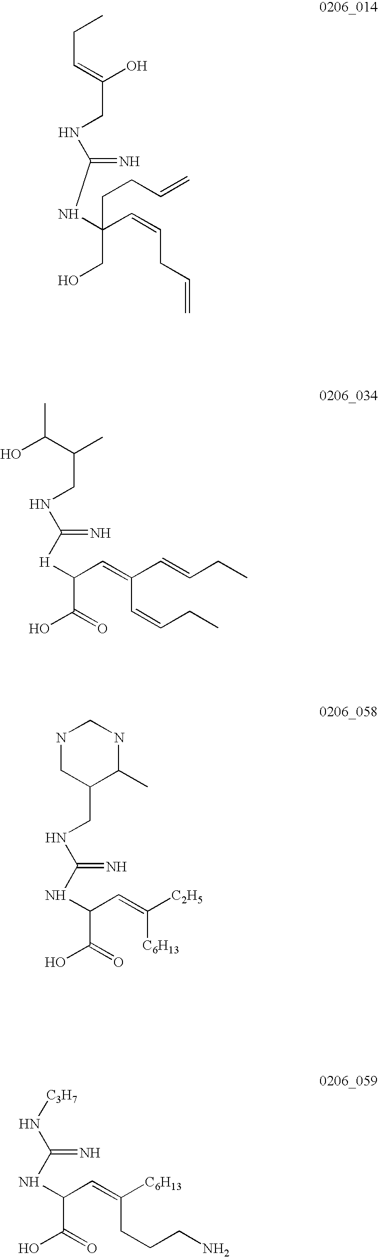

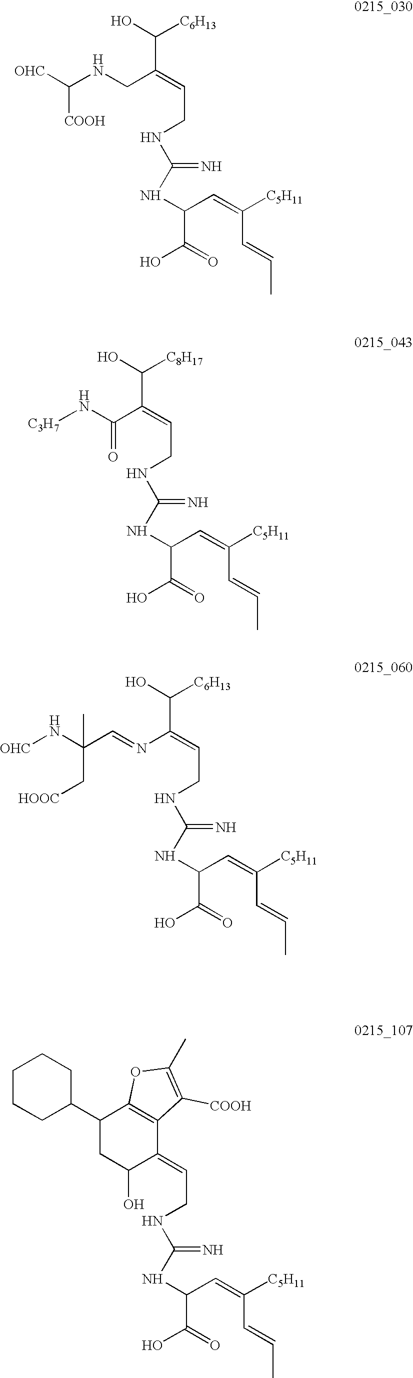

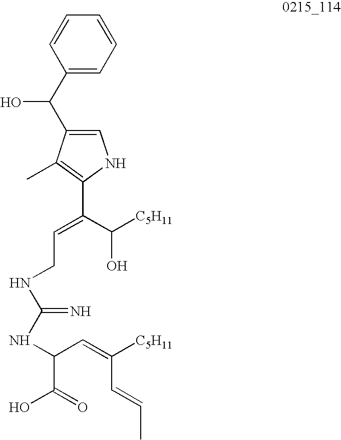

It is noted that the above compounds are characterized by various groups and substituents that correspond to the variables of the formulas listed above. It is intended to include each substituent and group within the definition of each variable, as if specifically recited therein.

In another preferred embodiment a pharmaceutically acceptable SOD aggregation inhibitor is a ligand capable of interacting with at least one beta barrel end thereby inhibiting SOD aggregation. S5 and S6 of SOD form the more exposed of the two barrel-ends and they are located at the opposite end to the dimeric interface. The surface of S5-S6 cleft is solvent exposed, but the geometric arrangement of the strands in spacefill-mode revealed regions that are partly buried enough for potential ligand design (FIG. 3A). Site characterization on the proposed target site indicated potential bindings in the vicinity of the residues 65-69, 74-81, 86-88, 95-99 and 102-103 (FIG. 3B). The stretch of the residues forms two regions of separate potential binding sites that are located near the beginning and the end of the barrel end. Therefore it may be possible to design a ligand that will span the length of the cleft while stabilizing the fold of the protein, which may achieve the earlier-described synergistic anti-aggregation effect on SOD.

Further extension of the logic that was used to derive the S5-S6 cleft target site yields S1-S8 cleft as another potential drug target site. However, S1-S8 cleft is significantly different in a few important instances, which may result in ligands that will work in significantly different fashion than those designed against S5-S6 cleft.

S1-S8 cleft in the native dimer of SOD is mostly buried by the dimeric interface, limiting the potential binding site to a very small region consisting of residues 7-11 and 146-147 (FIG. 4A). The site also lacks partly buried regions that are necessary for optimal binding of a ligand. Addition of the neighboring stretch 53-57 introduces some partly buried regions that may serve as anchor-sites, but stabilization of the cleft seems difficult without binding to the S1-S8 region. However, the site offers a distinct favorable feature when dimeric SOD is considered (FIG. 4B). In the native dimer, the clefts of the subunits form a cavity that may serve as an ideal binding site for a ligand through dimeric coordination. Such ligand binding is likely to stabilize the native dimer conformation while preventing the formation of an open-dimer. Our proposed mechanism of aggregation discussed in Example 2 and FIG. 9 as well as those of others (Ray, S. S., et al., Biochem. 43, 4899-4905 (2004) and Rakhit, R., et al., J. Biol. Chem. (2004)) then predict aggregation inhibition via stabilization of the native dimer.

The existence of structural protection motif for the barrel-ends was recently proposed for SOD, and the loss of the protection was argued to be critical in the process of aggregation (Khare, S. D., et al., J. Mol. Biol. 334, 515-25 (2003)). Furthermore, the ends of the barrel that were recognized as the potential site of aggregation (Khare et al. supra) coincide with the site of the intermolecular contact as proposed in our aggregation model described in Example 2. Therefore, ligands designed against the barrel-ends may have synergistic effects in preventing the aggregation of SOD by simultaneously stabilizing the intermolecular beta interaction (bonding) and destabilizing the intermolecular beta interaction (steric).

Therefore, in one preferred embodiment, a binding ligand capable of interacting with at least one beta barrel end and inhibiting aggregation of SOD comprises one or more of the following: (a) a chemical moiety capable of specifically binding at least one of SOD amino acids Asn 65, Leu 67, Arg 69, Glu 77, Lys 75, Pro 74, Asp 101 or Val 103; (b) a chemical moiety capable of specifically binding at least one of SOD amino acids Asn 65, Leu 67, Arg 69, Glu 77, Lys 75, Pro 74, Asp 101 or Val 103, and one or more amino acids in the S5-S6 cleft; (c) a chemical moiety capable of binding one or more SOD amino acids in the S5-S6 cleft; (d) a chemical moiety capable of binding one or more SOD amino acids located within 15 Å of the S5-S6 cleft; (e) a chemical moiety capable of binding one or more SOD amino acids located within 10 Å of the S5-S6 cleft; (f) a chemical moiety capable of binding an amino acid near the beginning and the near end of the S5-S6 cleft; (g) a chemical moiety capable of binding an amino acid near the beginning and the end of the S5-S6 cleft and wherein the chemical moiety spans the length of the S5-S6 cleft; (h) a chemical moiety capable of binding at least one of amino acids 74-82 and at least one of amino acids 102-103, (i) a chemical moiety capable of binding at least one of amino acids 86-88 and at least one of amino acids 95-99; and (j) a chemical moiety capable of binding at least one amino acid selected from amino acid residues 65-69, 74-81, 86-88, 95-99 and 102-103.

In another preferred embodiment, a binding ligand capable of interacting with at least one beta barrel end and inhibiting aggregation of SOD comprises one or more of the following: (a) a chemical moiety capable of specifically binding at least one of SOD amino acids Lys 9, Gly 147, Asp 11, Cys 57, Cys 146, Gly 56, and Asn 53; (b) a chemical moiety capable of specifically binding at least one of SOD amino acids Lys 9, Gly 147, Asp 11, Cys 57, Cys 146, Gly 56, and Asn 53, and one or more amino acids in the S1-S8 cleft; (c) a chemical moiety capable of binding one or more amino acids in the S1-S8 cleft; (d) a chemical moiety capable of binding one or more amino acids located within 15 Å of the S1-S8 cleft; (e) a chemical moiety capable of binding one or more amino acids located within 10 Å of the S1-S8 cleft; (f) a chemical moiety capable of blocking the S1-S8 cleft and preventing reduction of the Cys 157 to Cys 146 disulfide bond; (g) a chemical moiety capable of stabilizing the Cys 157 to Cys 146 disulfide bond by blocking the S1-S8 cleft; (h) a chemical moiety capable of binding at least one of amino acids 7-11 or 146-147, (i) a chemical moiety capable of binding at least one of amino acids 7-11 or 146-147 and at least one of amino acids 53-57; and (j) a chemical moiety capable of binding at least one of amino acids 53-57.

The present invention further provides a method for treating ALS, FALS or any other disease, particularly a SOD aggregation-mediated motor neuron disease in which inhibition of SOD aggregation is therapeutically desirable. The method comprises administering to the patient a therapeutically effective amount of a pharmaceutically acceptable SOD aggregation inhibitor, such as a SOD inhibitor designed in accordance with a method of the present invention. In one preferred embodiment, a SOD aggregation inhibitor of the invention is 4-pyrimidinecarboxylic acid. Even more preferably, a SOD aggregation inhibitor of the invention is orotic acid (vitamin B13) or any salt, ester or prodrug thereof.

Such treatment of disease includes methods of using a SOD aggregation inhibitor as an adjuvant or cotherapy in the treatment of ALS, FALS or any other disease or motor neuron disease in which inhibition of SOD aggregation is therapeutically desirable. In one embodiment, a pharmaceutically acceptable compound of the invention which inhibits SOD aggregation mediated by a Cys-111 amino acid of SOD is administered to a patient in need thereof.

A “therapeutically effective amount”, as this term is used herein, is an amount which results in partial or complete inhibition of disease progression or symptoms or prevention of the disease. Such an amount will depend, for example, on the size and gender of the patient, the condition to be treated, the severity of the symptoms and the result sought, and can be determined by one skilled in the art.

The invention further provides pharmaceutical compositions comprising one or more of the SOD aggregation inhibitors described above. Such compositions comprise a therapeutically effective amount of one or more SOD aggregation inhibitors, as described above, and a pharmaceutically acceptable carrier or excipient. Suitable pharmaceutically acceptable carriers include, but are not limited to, saline, buffered saline, dextrose, water, glycerol, ethanol, and combinations thereof. The carrier and composition can be sterile. The formulation should suit the mode of administration.

Suitable pharmaceutically acceptable carriers include but are not limited to water, salt solutions (e.g., NaCl), alcohols, gum arabic, vegetable oils, benzyl alcohols, polyethylene glycols, gelatin, carbohydrates such as lactose, amylose or starch, cyclodextrin, magnesium stearate, talc, silicic acid, viscous paraffin, perfume oil, fatty acid esters, hydroxymethylcellulose, polyvinyl pyrrolidone, etc. The pharmaceutical preparations can be sterilized and if desired, mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrrolidone, sodium saccharine, cellulose, magnesium carbonate, etc.

The composition can be formulated in accordance with the routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water, saline or dextrose/water. Where the composition is administered by injection, an ampoule of sterile water for injection or saline can be provided so that the ingredients may be mixed prior to administration.

The pharmaceutical compositions of the invention can also include an agent which controls release of the SOD aggregation inhibitor compound, thereby providing a timed or sustained release composition.

The SOD aggregation inhibitor can be administered subcutaneously, intravenously, parenterally, intraperitoneally, intradermally, intramuscularly, topically, enterally (e.g., orally), rectally, nasally, buccally, sublingually, vaginally, by inhalation spray, by drug pump or via an implanted reservoir in dosage formulations containing conventional non-toxic, physiologically acceptable carriers or vehicles. The preferred method of administration is by oral delivery. The form in which it is administered (e.g., syrup, elixir, capsule, tablet, solution, foams, emulsion, gel, sol) will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. The compounds and agents of this invention can be administered together with other biologically active agents, such as analgesics, anti-inflammatory agents, anesthetics and nucleoside-based drugs.

In a specific embodiment, it may be desirable to administer the agents of the invention locally to a localized area in need of treatment; this may be achieved by, for example, and not by way of limitation, local infusion during surgery, topical application, transdermal patches, by injection, by means of a catheter, by means of a suppository, or by means of an implant, said implant being of a porous, non-porous, or gelatinous material, including membranes, such as sialastic membranes or fibers. For example, the agent can be injected into the joints.

### EXAMPLES

### Example 1

### Cys 111 is involved in Oxidative Damage-induced Aggregation of SOD

This experiments shows that Cys 111 is involved in the oxidative damage-induced aggregation of SOD. Wild type SOD and three pathogenic mutants (A4V, G37R and G85R) were used in the experiment. Mean survival associated with A4V, G85R and G37R are 1, 6 and 18 years, respectively. The sulfur of Cys 111 was blocked with various sulfhydryl-blocking reagents, such as iodoacetate and dithionitro benzoic acid (DTNB). Iodoacetate treatments of proteins with exposed Cysteines results in the acetylation of sulfhydryl groups. DTNB selectively attacks accessible sulfhydryl groups in proteins and attaches one 2-nitrobenzoate group to the exposed Cysteine via a disulfide bond.

SOD expression and purification. The SOD expression vectors were a gift from Joseph Beckman (Oregon State University). The SOD cDNA was cloned into a PET21 vector between the BAMHI and NcoI sites and transformed into Escherichia coli strain BL21 pLysS-competent cells as previously described [9]. Cells were grown in Luria-Bertani (LB) media to an OD₆₀₀ of 0.8, at which time isopropyl-β-D-thiogalactoside was added to a final concentration of 0.3 mM. After 1 h of induction, cells were collected by centrifugation (8000 g) at 4° C. and frozen at −80° C. The frozen cells were resuspended in 50 mM phosphate buffer (pH 7) and 150 M NaCl, and then lysed by four cycles of sonication with 30 s bursts with a 1 min interval of cooling in ice. The purification of SOD was carried out as previously described in Hayward et al., J. Biol. Chem., 277 (2002) 15923-15931, incorporated herein by reference.

Demetallation of SOD. Purified SOD was demetallated as previously described in Sutter et al., Protein Expr. Purif., 19 (20000) 53-56, incorporated herein by reference. SOD samples were then dialyzed against 20 mM tris(hydroxymethyl)aminomethane hydrochloride (Tris) buffer at pH 7.8. All SOD samples were prepared fresh just before the experiment.

Unmodified SOD aggregation. SOD aggregation of SOD with an umodified Cys-111 residue was achieved by mixing 10 μM SOD with 0.8 mM copper sulfate (CuSO₄) and 10 mM hydrogen peroxide (H₂O₂) in 20 mM Tris at 37° C. (pH 7.4). SOD aggregation of SOD modified at the Cys-111 residue.

Modified (Blocked Cys-11 Residue) aggregation. Treatment with iodoacetate and DTNB was carried out at 0.73 mg/ml demetallated SOD, 20 mM Tris, 20 mM phosphate with 10 mM modifying reagent in each at pH 7.4 and room temperature for one hour. In the case of DTNB treatment, both protein solution and the buffer were nitrogen fused prior to the reaction. Modified SOD samples were then dialyzed against 20 mM Tris buffer at pH 7.8 in room temperature. All of the modified SOD samples were prepared fresh just before inducing oxidation with copper (0.8 mM) and hydrogen peroxide (10 mM).

Kinetic Studies of modified and unmodified SOD aggregation. To monitor the kinetics of aggregation, the SOD solution was equilibrated in a quartz cuvette at 37° C. for 5 min within the sample chamber of a Hitachi F-4500 fluorimeter, at which point a freshly made solution containing the CuSO₄, H₂O₂, and Tris was added. Using a small stir bar, the solution was mixed for 5 s, and the light scattered at a 90° angle was immediately monitored for 1 h at an excitation and emission wavelengths of 350 with 2.5 nm slit width. Afterwards, the aggregation mixture was centrifuged at 18,000 g and the SOD aggregate was then resuspended in 50 μL fresh water for storage at room temperature.

Atomic force microscopy and the sample preparation. Aggregated pellet resuspended in 50 μL fresh water was sonicated for 10 min. The suspension was then spun at ˜18,000g for 30 s. A 20 μL drop of the supernatant was placed onto a freshly cleaved mica and it was dried by slow evaporation over 3 h followed by 30 min vacuum-drying before imaging using tapping mode AFM in a Multimode Nanoscope IIIa instrument (Digital Instrument/Veeco Metrology Group, Santa Barbara, Calif.). Silicon tips with a radius of ˜10 nm (nominal spring constant=31-43 N/m, resonant frequency=160-210 kHz) were used as probes.

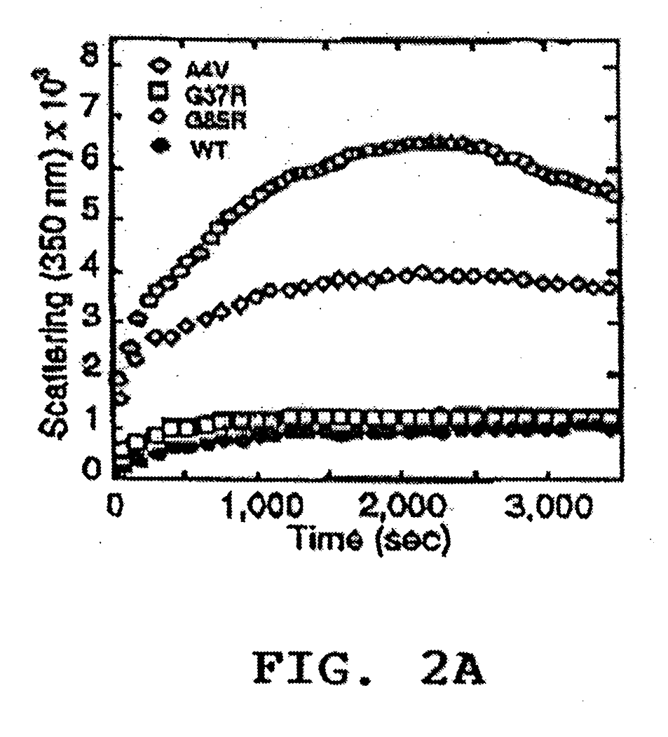

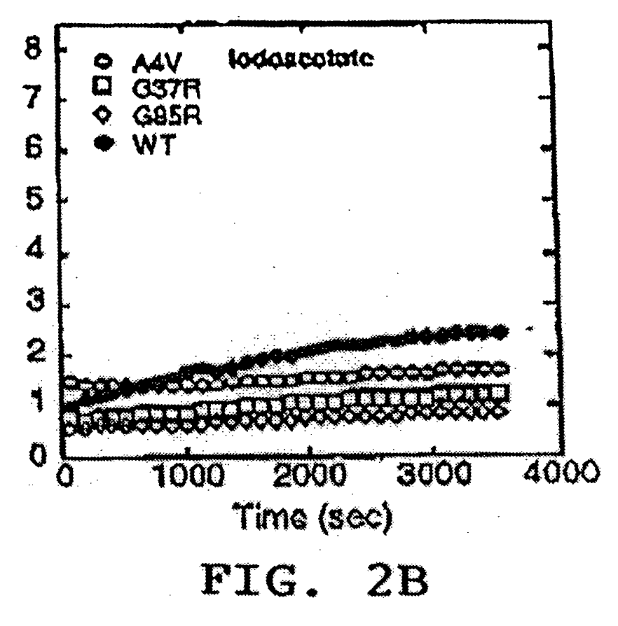

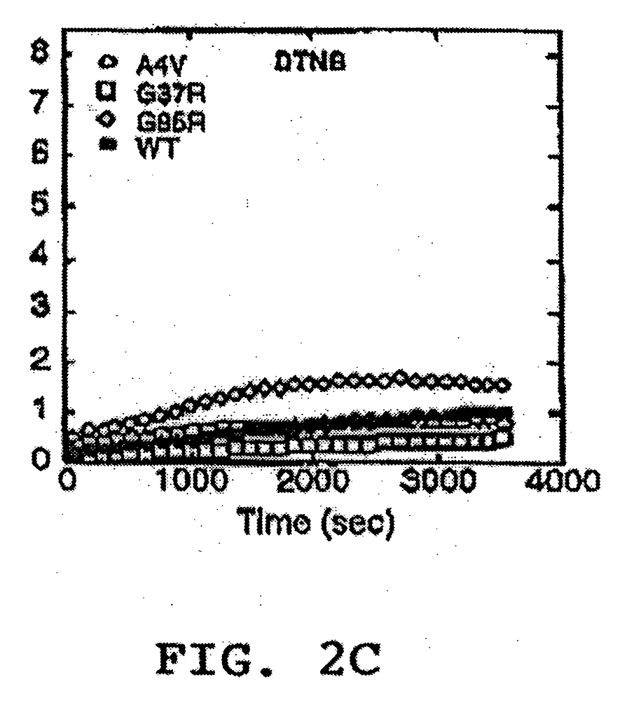

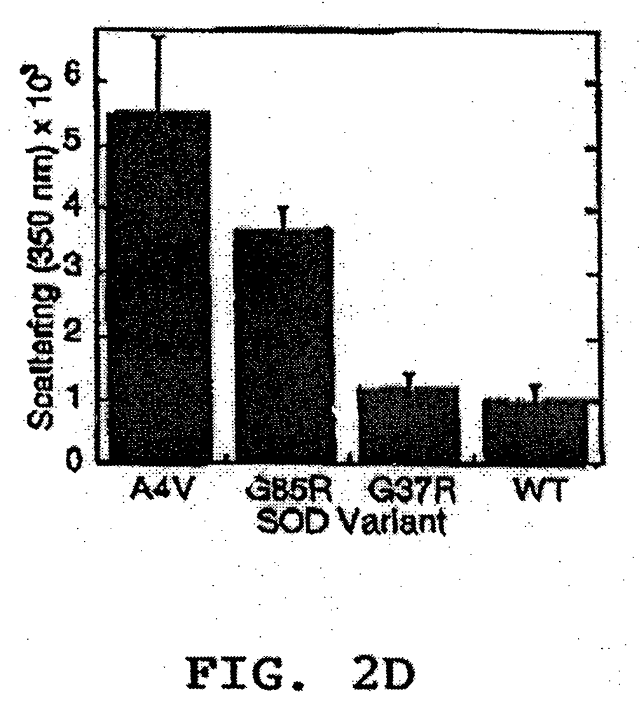

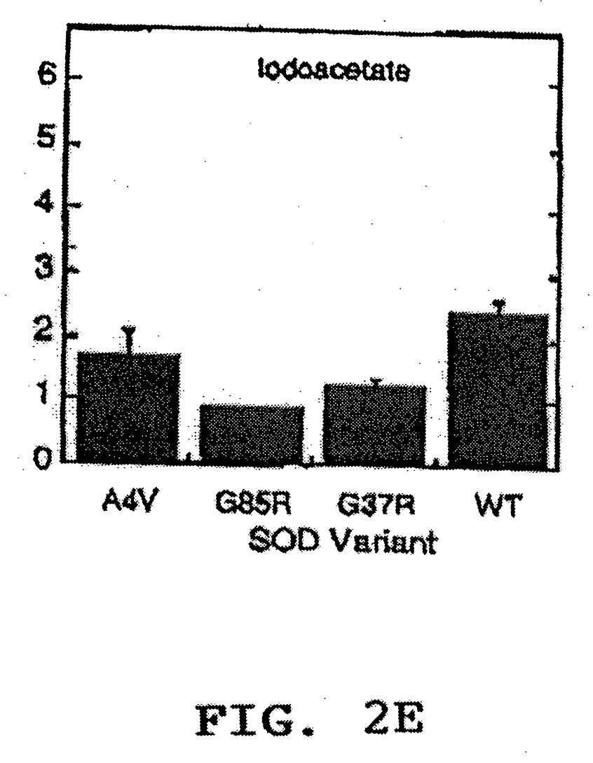

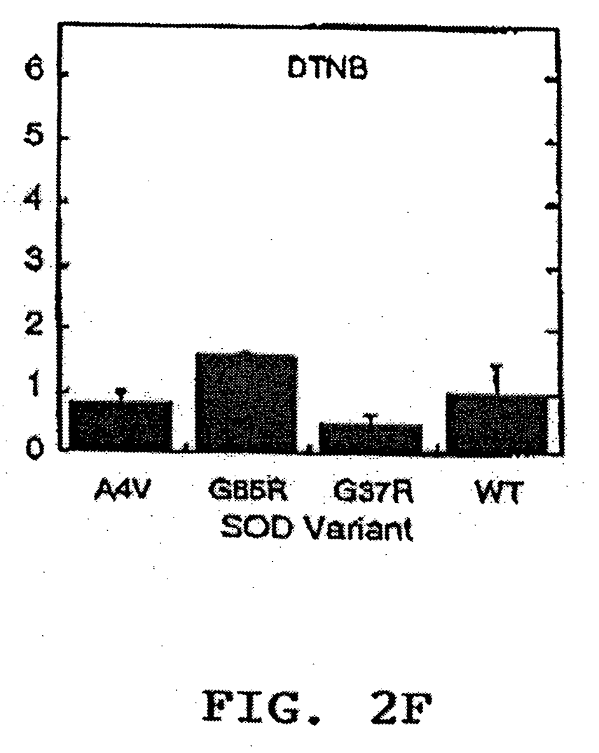

Results and Discussion. FIG. 2 shows the inhibition of Cys-111-blocked SOD aggregation. FIG. 2A shows the aggregation of apo SOD in the absence of Cys 111 modification, and FIG. 2 B and C show the aggregation of SOD after treatment with iodoacetate or DTNB, respectively. FIG. 2D shows the assessment of final aggregation extent by apo SOD1 in the absence of Cys 111 modification, and FIGS. 2E and 2F show the aggregation of SOD after treatment with iodoacetate or DTNB, respectively. These results indicate that copper-induced oxidation of metal-depleted SOD causes its in vitro aggregation into pore-like structures and that such aggregation is inhibited in the presence of a sulfhydryl blocking reagent. Because these toxic pores have been implicated in the pathogenic mechanism of other neurodegenerative diseases, these results indicate that compounds capable of blocking all or a portion of the critical region of SOD, may be useful in the treatment of ALS, FALS and related diseases in which the inhibition of SOD aggregation is desirable.

### Example 2

### Model for SOD Aggregation

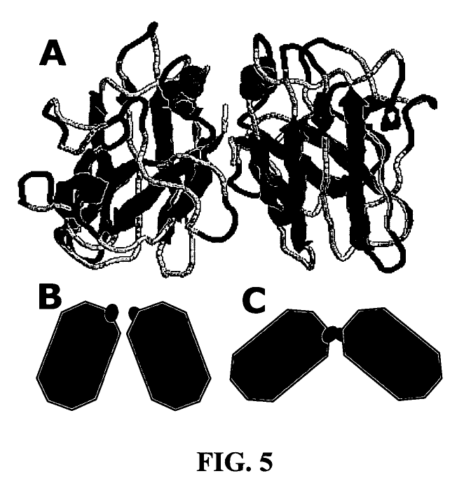

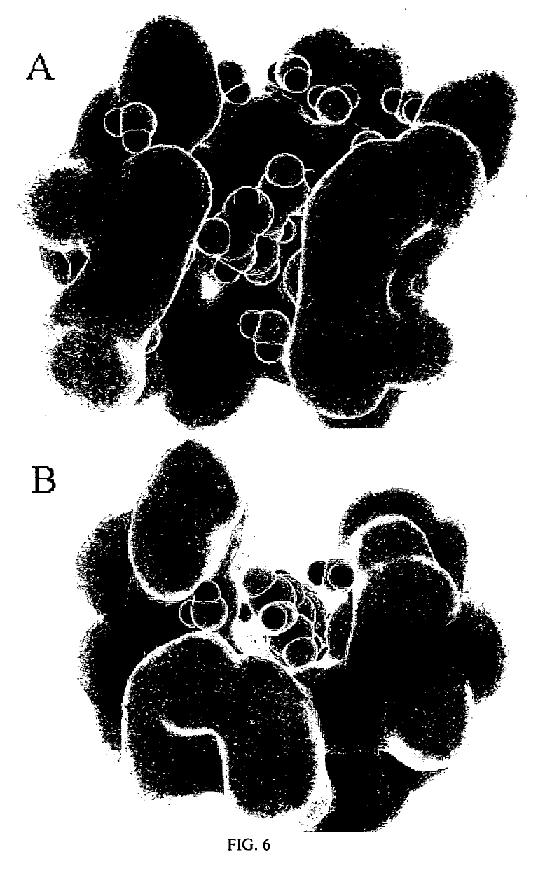

Recent advances in the studies of SOD aggregation have suggested the involvement of monomeric intermediate (Ray, S. S., et al., Biochemistry 43, 4899-4905 (2004), Rakhit, R., et al., J. Biol. Chem. (2004) and Khare, S. D., et al., Proc. Natl. Acad. Sci. USA 101, 15094-9 (2004)) and the loss of the metal cofactors (Khare et al. supra) as the critical events leading to the aggregation of SOD. On the contrary, suggestions of dimeric intermediates in the process of aggregation were also made (DiDonato, M., et al., J. Mol. Biol. 332, 601-15 (2003) and Hough, M. A., et al., Proc. Natl. Acad. Sci. USA 101, 5976-81 (2004)). Specifically, Hough and colleagues reported significant distortions in the dimeric conformation of A4V and I113T mutants, which could only be observed in solutions (Hough et al. supra). Briefly, Hough et al compared the crystal structures of WT, A4V and I113T mutant SOD with those observed in solution using small angle X-ray scattering (SAXS), and found different solution structures arising in the mutants tested. According to the report, the WT SOD showed similar structures in both crystal and in solution with its subunit beta-barrels of WT SOD aligned nearly parallel to the axis of dimerization (FIG. 5, Panel A and B). A4V and I113T mutants, on the other hand, were shown to adopt conformations that resulted in a wider angle between the two axes of beta-barrels (FIG. 5, Panel C). For convenience, we will refer to the distorted conformation as “open.”

The open conformation of mutant SOD possesses distinct structural features in comparison to the WT-like conformation in two main aspects. First, it increases exposure of both beta-barrel ends. Protection of the edges was suggested to be critical in preventing the aggregation of SOD (Khare, S. D., et al., J. Mol. Biol. 334, 515-25 (2003)). Second, it forms new dimeric contacts, partly burying the crevice near the Cysteine 111 residue (Cys111). This finding was of special interest to us since a covalent modification at Cys111 was shown to reduce oxidative aggregation of WT SOD (de Beus, M. D., et al., Protein Sci. 13, 1347-55 (2004)). Hence, to better understand the mechanism of SOD aggregation, we decided to investigate the role of the Cys111 region and the open conformation in the aberrant self-assembly of SOD using apo SOD (Khare et al. supra).

Extent of Dimer Opening is Related to the Oligomerization Propensity of Apo SOD.

The previously reported open-dimer conformation of A4V and I113T SOD mutants (Hough et al. supra) buries the Cys111 region, which contains the only surface-exposed Cysteine residue of SOD. Therefore, we attempted to assess the extent of the dimeric structural deformation in different mutants and WT apo SOD by studying the Cysteine accessibility via Ellman's Assay (Ellman, G. & Lysko, H., Anal. Biochem. 93, 98-102 (1979)). Our experimental results showed that some of the SOD mutants had significantly lowered Cysteine accessibility than WT, with the exceptions of dimeric interface mutants A4V and L144F. The finding that G37R, D90A and G85R had significantly reduced Cys111 accessibility compared to WT SOD suggests that they adopt the open dimer conformation as found in A4V and I113T (Hough et al. supra). The increased Cysteine accessibility in A4V may be explained by its increased dissociation into monomers (Ray, S. S., et al., Biochemistry 43, 4899-4905 (2004)).

While studying the glutaraldehyde cross-linking (GCL) efficiency of apo SOD, we came across the serendipitous finding that different SOD variants showed different amounts of oligomeric bands on SDS-PAGE. Upon a more careful comparative study of their relative oligomerization extent during GCL, we noticed an inverse-relationship between Cys111 accessibility and the oligomerization propensity with the notable exception of A4V. Considering the fact that Cys111 accessibility reflects the extent of dimer opening, this finding suggested the possibility that the extent of dimer opening might be responsible for the increased oligomerization propensity. The possibility appeared to be consistent with our previous finding that oxidative modification at Cys111 reduced the aggregation of holo WT SOD (de Beus et al. supra) since the modification would sterically hinder the formation of open-dimers. Therefore, we decided to study the oxidative aggregation of apo SOD with different covalent modifications at Cys111 to further probe the importance of the Cys111 region and the deformations in dimeric SOD.

Covalent Modifications to Cys111 Modulate Cu-H₂O₂-induced Oxidative Aggregation of Apo SOD.

Using A4V, G37R and WT SOD, we decided to test carboxymethyl modification at Cys111, which should disrupt the formation of “opened-dimer” conformation through steric and charge-repulsion effects. As suspected, all modified apo SOD variants showed noticeable reduction in the extent of aggregation, supporting the hypothesis that the open-dimer conformation contributes to the aggregation propensity. Methylation of Cys111 also resulted in the inhibition of aggregation during the early phase (introduction of lag-phase), providing further support for the role of Cys111 region during oxidative aggregation of SOD. Next, we decided to test the effect of persulfide modification at Cys111 (addition of a sulfur atom at the end of Cysteine sulfhydryl group) on the aggregation propensity of SOD. The persulfide group is known to be more prone to disulfide bond formation due to its enhanced nucleophillic attacking ability (Iciek, M. & Wlodek, L., Pol. J. Pharmacol. 53, 215-25 (2001)), and thus SOD dimers should have greater probability of forming Cys111-Cys111 disulfide bond under oxidative stress. Consequently, formation of a Cys111-Cys111 inter-subunit disulfide bond would favor the open-dimeric conformation, resulting in increased aggregation propensity. Our oxidative aggregation experiments on persulfide modified apo SOD confirmed our hypothesis by showing noticeably enhanced aggregation in contrast to their non-modified and carboxymethylated counterparts. Altogether, the results suggest that the open-dimer formation may be critical for the oxidative aggregation of apo SOD.

Methylation at Cys111 Inhibits Non-oxidative Fibrillar Aggregation of Apo SOD During pH-Induced Refolding.

While studying the pH-induced refolding of SOD, we noticed aggregation of apo SOD that could be detected by light scattering. Thus, we decided to investigate it as a non-oxidative aggregation method using apo WT, G37R and A4V. Morphology study of the resulting aggregates through atomic force microscopy (AFM) revealed mainly spherical aggregates in WT, but G37R and A4V showed highly ordered aggregates. Specifically, the mutants showed fibrillar aggregates with minimum width of 6˜7 nm that showed no thioflavin-T binding. Pore-like aggregates similar to those reported previously in oxidation aggregation (Chung, J., et al., Biochem. Biophys. Res. Commun. 312, 873-6 (2003) and Ray et al. supra) could not be found.

In order to investigate the involvement of open-dimers in the pH-refolding aggregation of apo SOD, we decided to introduce methyl modification to Cys111 since methylation is less likely than carboxymethyl modification to disrupt refolding. Surprisingly, methylated apo SOD showed complete inhibition of aggregation in all three SOD (WT, G37R and A4V) tested. This result suggests the importance of the contacts made near the Cys111 region in the pH-induced refolding aggregation.

Defective pH-Induced Refolding of Mutant SOD.

Our finding in the previous section that apo G37R and apo A4V aggregated into 7 nm-fibrillar aggregates while the apo WT SOD did not during the pH-induced refolding suggested the possibilities of misfolding in the mutants. Indeed, dynamic light scattering (DLS) of the apo SOD variants before and after the pH-refolding revealed changes in the hydrodynamic radius (Rh) that were suggestive of defective refolding in the pathogenic mutants G37R and A4V. Briefly, apo WT showed the same Rh before and after the pH-induced refolding, which is consistent with our earlier report of folding reversibility of WT SOD (de Beus et al. supra). The mutants, on the other hand, showed significant increase in Rh after refolding, indicating defective refolding.

Cysteine-Cross-Linked SOD Dimer and Trimer are Implicated in the pH-Refolding Aggregation.

Cysteine-cross-linking of SOD during thermal (Stathopulos, P. B., et al., Proc. Natl. Acad. Sci. USA 100, 7021-6 (2003)) and chemical denaturation (Assfalg, M., et al., J. Mol. Biol. 330, 145-58 (2003)) has been known to be associated with aggregation. To see if the same holds true for pH-induced refolding, we decided to filter the refolded samples with fine filters (0.22 μm and 20 nm, Whatman) and assess the concentration of oligomer through Westernblot. Filtration of the samples through 0.22 μm filters caused no apparent changes in the composition of samples. In contrast, Westernblot of the corresponding samples further revealed significant removal of Cysteine-cross-linked dimer and trimers of all SOD variants in the pH-refolded samples. Specifically, A4V SOD showed the biggest contrast with almost all cross-linked dimers and trimers gone in the filtered portion. In contrast, the removal of cross-linked species upon filtration through 20 nm filter was accompanied by significant reduction in concentration of all refolding samples. These findings suggested greatly enhanced aggregation propensity in the Cysteine cross-linked dimers and trimers of SOD.

Addition of Salt Induces the Formation of Trimeric and Oligomeric SOD

Aggregation of SOD was previously suggested to be promoted by high-salt conditions (DiDonato et al. supra). Hence, we decided to assess the effects of salt on the oligomerization state of apo WT SOD using GCL and SDS-PAGE. When the NaCl concentration was raised in 20 mM phosphate buffer, increasing densities in both trimeric and oligomeric SOD bands were observed. Interestingly, the appearance of the trimeric/oligomeric bands coincided with the decreasing monomeric band. Use of potassium phosphate gradient also promoted the formation of trimers/oligomers, although trimers were not as abundant as in the NaCl gradient. The population of GCL cross-linked dimers showed relative little change over the increasing gradient of phosphate while the monomeric band progressively decreased. Together, the two results suggest the presence of a dimeric conformer that is difficult to cross-link and that is the precursor to the monomeric (“M”) and oligomer bands (“O”).

Dimer Opening is Implicated in the Oligomerization of apo SOD.

We have shown evidence that suggests different oligomerization propensities of different apo SOD variants using GCL, and the fact that these oligomers are not observed in sizable population without GCL (Ray et al. supra) suggests that they are in equilibrium with the dimeric conformation. Additional evidence of the reversible oligomerization could be found in the GCL experiment of apo WT SOD with varying salt concentrations. The experiment showed that even the apo WT SOD could be induced to have higher oligomerization propensity, perhaps through shifting of the equilibrium. Furthermore, the results suggest that that the dimeric conformers that failed to cross-link may be the precursors to the non-native oligomers. Hence, understanding the structure of the glutaraldehyde cross-linked dimeric SOD may provide an explanation for the process of reversible oligomeriation.

GCL of proteins is known to work by cross-linking the terminal amines of lysine residues on the protein surface (Hardy, P. M., et al., J. Chem. Soc. [Perkin 1] 9, 958-62 (1976)). SOD dimer has a total of 22 lysine residues, and yet the Lys9 pair is the only pair of lysines that are within 10 Å from each other and capable of inter-subunit cross-linking. The inter-subunit cross-linking by Lys9, however, may be inhibited competitively by Lys36. Therefore, the formation of open-dimer would separate the lys9 residues, thereby reducing the chance of inter-subunit cross-linking. This suggests that the ability to form open dimers may be closely related to the propensities of apo WT SOD to form trimeric and oligomeric species. Not surprisingly, our multi-mutant studies on the Cys111 accessibility and GCL-induced oligomerization also suggested a close relationship between the opening of dimer and the oligomerization propensity during GCL. All in all, our results from different experiments strongly suggest the open dimer as the common culprit in the reversible oligomerization process of apo SOD.

Aggregation-Prone Open Dimer may be on the pH-Induced Folding Pathway.

The open-dimeric conformation in mutants of SOD are observed only in solution (Hough et al. supra). In contrast, all of the mutants and WT SOD studied so far have shown WT-like conformation (Parge, H. E., et al., Proc. Natl. Acad Sci. USA 89, 6109-6113 (1992), DiDonato et al. supra, Hough et al. supra and Strange, R. W., et al., J. Mol. Biol. 328, 877-91 (2003)) in crystal structures. This difference in the structures of mutant SOD observed was proposed to be due to stabilizing effects of the salts used during crystallization (Hough et al. supra), which suggests a possible folding-pathway connection between the open dimer and the native dimer. And if so, an important question arises: Is the open-dimer on the refolding pathway to the native WT-like conformation? The observation that methylation of Cys111 inhibited pH-refolding induced aggregation in all variants of apo SOD suggests close-interaction at the Cys111 crevice during the refolding process. This interaction at Cys111 crevice during the refolding process appears to be a part of the native refolding process. Specifically, given the fact that apo WT refolded back to the conformation with the same radius of hydration (Rh) it is apparent that apo WT was on-pathway to its correct fold when the close-interaction at Cys111 crevice was taking place. This suggests a possibility that apo WT SOD was in open conformation prior to reaching its final conformation. Folding of WT SOD was previously reported to be somewhat reversible when a pH change of 7→2→7 takes place (de Beus et al. supra). Therefore, if the open dimer is truly on the folding pathway of WT SOD, changes in the Cys111 crevice should also affect the unfolding of SOD. Indeed, WT SOD with persulfide/polysulfide modification at Cys111 was previously shown to have a significantly reduced rate and irreversible kinetic unfolding (de Beus et al. supra), suggesting the presence of the open dimer on the unfolding path of WT SOD.

Understanding the Dimer-Opening and its Implications to the Aggregation Mechanism of SOD.

Our experiments describing oligomerization propensity, oxidative aggregation and pH-refolding induced aggregation of apo SOD showed that the three modes of SOD aggregation are consistently associated with a single character commonly identified in the pathogenic mutants tested: formation of open-dimers. Thus, better understanding of the responsible conformer may help explain the mechanism underlying the aggregation. As described earlier, the main difference between native dimers of WT SOD and open-dimers of the mutants is the rotation of subunits leading to partial opening of the native dimeric interface and closing of the Cys111 crevice. Two subtle consequences result from the dimeric structural deformation, which may help explain the increased aggregation propensity of the open-dimeric conformation. The first consequence is that SOD starts to expose both of its beta-barrel ends, whose protection was proposed to be critical in the process of aggregation (Khare et al. supra). Secondly it is important to note that the beta-strands of SOD start to align perpendicular to the axis of dimerization, which may allow for stacking of open-dimers by the edges of the beta-barrels. Therefore, if the protection of the edges of beta-barrel is compromised, open-dimers of SOD may readily associate with others by the beta-barrel ends. Thus, under certain conditions, one can expect to observe fibrils with widths of SOD dimer. We have morphological evidence of the corresponding fibril structure from apo A4V with an approximate width of 7 nm. Considering the fact that the height of an SOD subunit is roughly 3.3 nm (Parge et al. supra), an open-dimer would have an approximate length of 6.5 nm, which is consistent with our data.

Both pH-refolding Induced Fibril Formation and Pore-like Aggregates may Arise from Open Dimers

The aggregates produced from pH-refolding of apo G37R and A4V showed fibrillar tangles with minimum widths of approximately 7 nm by AFM. On the contrary, we were unable to find similar morphology in apo WT samples, which suggested to us that the fibrils might have come from open-dimers common to both G37R and A4V. Furthermore, the fact that methylation at Cys111 inhibited the pH-refolding induced aggregation appears to be suggestive of the involvement from open dimers in the process. Pore-like aggregates previously reported (Chung et al. supra and Ray et al. supra), on the other hand, needed a bit of math for clarification.

Dimension analysis of the pore-like aggregates from the oxidative aggregation previously reported (Chung et al. supra) showed an outer diameter of ˜20 nm and an inner diameter of ˜5 nm. Therefore, the width of the fibril forming the pore-like aggregate is roughly (20−5)/2=7.5 nm, which is consistent with the approximate dimension of the 7 nm fibril we have observed. Furthermore, our ability to modulate the oxidative aggregation propensity through two different modifications at Cys111 supports the view that the oxidative aggregation leading to the formation of pore-like aggregates is also mediated by open-dimers. One big question still remains: how could it be that the two different types of aggregates (oxidative vs pH) that occurred exclusively from one another were composed of the same building blocks? We believe that the abundance of the 7 nm fibrils may be the determining factor for the ultra structure of the final aggregates. That is, if the fibrils were readily available, they would be kinetically more likely to make inter-fibril contacts and form tangles such as those found in pH-refolding induced aggregation. In contrast, if the availability of the fibrils were low, it might be more advantageous kinetically for the fibrils to coil into the pore-like structures (Chung et al. supra and Ray et al. supra). This suggests that under normal physiological conditions where aggregates of SOD may not be as readily available, pore-like aggregates are more likely to occur than fibril tangles. It is also worth noting that pores of different sizes were reported with A4V mutant SOD upon incubation at neutral pH and at RT (Ray et al. supra). This finding is consistent with the idea that fibrils of different lengths can coil to form pores of different sizes, which may contribute to the inherent toxicity associated with each mutant.

A Model for SOD Aggregation Proposed

Given the scope of our findings and the works of others (DiDonato et al. supra, Ray et al. supra, Rakhit et al. supra, Khare et al. supra, Hough et al. supra and Khare et al. supra), we propose a general mechanism of SOD aggregation that will lead to ordered aggregate structures (FIG. 9).

We also attempt to make a more general aggregation mechanism by including the descriptions for the thioflavin-T positive aggregates reported by DiDonato et al., supra, including fibrils with ˜3.5 nm width as well as channel-like aggregates. Under denaturing conditions such as low pH with EDTA, partially denatured apo monomers may aggregate by the ends of their beta-barrel, forming fibrils with 3.5 nm (DiDonato et al. supra and Khare et al. supra).

The proposed aggregation model suggests three potential target sites for drug design: Cys111 crevice and the two ends of the beta-barrel. The current study has shown direct evidence of reduction in both oxidative and non-oxidative aggregation through covalent modifications at Cys111, and we are currently working on finding small ligand against the region. A ligand that binds to the two ends of the beta-barrel may inhibit both monomeric and dimeric aggregation by protecting the ends of the beta-barrel (Khare et al. supra), causing steric hindrance against the formation of inter-subunit beta contacts.

Potential Toxicity of SOD Aggregates Proposed

Our demonstration of the ability of apo SOD to form fibrils as well as pore/channel aggregates raises the question of toxicity associated with the morphologies. Is it the fibrils or the pore/channels that are toxic? We tried to find plausible explanation for this question from the clinical and model studies on ALS. Mitochondria serve many important roles in nerve cells, and one of their central functions is buffering of intracellular calcium level, which was shown to be compromised in the cells expressing mutant SOD (Carri, M. T., et al., FEBS Lett. 414, 365-8 (1997)). Furthermore, a number of model studies (Kong, J. & Xu, Z.,, J. Neuroscience 18, 3241-3250 (1998), Jaarsma, D., et al., Acta. Neuropathol. (Berl) 102, 293-305. (2001), Higgins, C. M., et al., J. Neurosci. 22, RC215 (2002) and Higgins, C. M., et al., BMC Neurosci. 4, 16 (2003)) have long identified mitochondria as among the first organelle to be affected by the toxicity of mutant SOD, and the aggregation of mutant SOD in the inter-membrane space of mitochondria was proposed to be responsible for mitochondrial dysfunction (Higgins et al. supra). More specifically, association of SOD aggregates with inner and outer mitochondrial membranes were shown to be responsible for the onset of mutant SOD toxicity. Thus, given the fact that SOD can aggregate to pore/channel-like aggregates (Chung et al. supra and Ray et al. supra), one could deduce that SOD aggregates may function as pores that permeate the mitochondrial membranes. In that sense, it is interesting to note that possible involvement of mitochondrial permeation transition pore (MPTP) was proposed (Bendotti, C., et al., J. Neurol. Sci. 191, 25-33 (2001)), even though the actual involvement of the permeation pore system turned out to be false (Higgins et al. supra).

The fact that mitochondrial dysfunction plays a central role in the pathogenesis may also provide some insights to the motor neuron selectivity in ALS. Neurons are characterized by high density of mitochondria due to their energy requirements for maintaining their membrane potential (Levitan, I. B. & Kaczmarek, L. K., “The neuron: cell and molecular biology,” (Oxford University Press, Oxford ; New York, 2002)). Given the fact that larger surface area implies greater energy requirements for maintaining the membrane potential in neurons, motor neurons would have the highest energy demand due to their exceptional length. Consequently, they would require greater number of mitochondria to meet their energy demands (Miller, R. J., Trends Neurosci. 15, 317-9 (1992) and Wong-Riley, M. T., Trends Neurosci. 12, 94-101 (1989)), thereby increasing the risk of SOD aggregation-mediated toxicity. In this context, it is interesting to note that lumbar (lower back), but not cervical (neck) motor neurons, were shown to have accumulation of SOD aggregates in transgenic mice model studies (Turner, B. J., et al., Neurosci. Lett. 350, 132-6 (2003)), suggesting a relationship between the length of neurons and accumulation of SOD aggregates. Selective survival of small motor neurons in the ventral root of the degenerated spinal cord in mice model studies was also reported (Vanden Noven, S., et al., Exp. Neurol. 123, 147-56 (1993), Kong, J. & Xu, Z., J. Comp. Neurol. 412, 373-80 (1999), Yawo, H., J. Neurosci. 7, 3703-11 (1987) and Standler, N. A. & Bernstein, J. J., Exp. Neurol. 75, 600-15 (1982)), further supporting the idea.

### Example 3

### Inhibitory Effect of Orotic Acid on the Aggregation of SOD In Vitro

The scope of the current study that led to the discovery of anti-aggregation effect of orotate was based the inventor's aggregation model involving the open dimers (FIG. 9) as discussed in Example 2. Thus, the inventor's experimental evidence suggesting the inhibition of in vitro oxidative aggregation provides supports for the proposed mechanism of aggregation. The subtle observation that orotate showed different level of anti-aggregation activity in different variants of SOD with different extent of dimer opening suggests that the aggregation inhibition is dependent on the organization of the Cys111 crevice. Taken together, these inhibition data suggest binding of orotate to the critical region of the Cys111 crevice, and the resulting inhibition of oxidative aggregation in vitro.

Orotate is characterized by its numerous sites for potential hydrogen bonding (Formula 2), which may be deemed as a sign of less binding specificity. Furthermore, the fact that the theoretical binding of orotate to the Cys111 crevice is driven by desolvation energy may indicate possible non-specific interaction through hydrophobic interaction. However, these features may be an advantage in binding to our target site, Cys111 crevice. The Cys111 crevice was suggested to be different from mutant-to-mutant. Hence, the ability of orotate to stabilize the binding through desolvation energy and to form many possible combinations of hydrogen bonding networks and salt-bridges may be the key to its ability to bind Cys111 crevice in various conformations.

There may be some site-specific interactions between Cys111 and orotate. We speculate that the distance between the salt-bridge forming carboxylate group and the hydrogen bond forming dioxypyrimidine group, as well as the triangular H-bond donor arrangement on the dioxypyridine ring may be the key to the selectivity question, as the distance between the two groups is optimal for simultaneous coordination of the orotate-R115 salt bridge and the hydrogen bonding network about Cys111.

Orotic acid is a common vitamin that is widely available, and current work shows some promising results that may suggest the potential of orotic acid as a therapeutic agent for SOD-mediated ALS. Interestingly, Morifuji and Aoyama, supra, recently reported reduction in the Cu/Zn superoxide dismutase mRNA level and corresponding reduction in SOD activity in rat liver upon administration of dietary orotic acid (Kong et al. supra). If similar expression control for Cu/Zn SOD exists in human motor neurons, administration of orotic acid may help SOD toxicity in ALS by two means: inhibition of toxic aggregation and down-regulation of Cu/Zn SOD at genetic level. An additional advantage of orotic acid as a treatment for ALS is that orotate can freely diffuse across cell membranes, including that of the blood-brain barrier (Jaarsma et al. supra). Furthermore, administration of dietary orotate has been already shown to improve memory and synaptic long-term potentiation in mouse models (Jaarsma et al. supra), as well as the conditions of cardiomyopathic hamsters (Higgins et al. supra). Hence, the use of anti-oxidants such as SOD-mimetics (Higgins et al. supra) and orotic acid may be a promising approach for the treatment of SOD-mediated ALS. Chemical derivatives of orotate and uracil compounds that improve the stability in physiological conditions of the compounds and impart other desirable therapeutic qualities to the compounds are also contemplated herein.

The patent and scientific literature referred to herein establishes the knowledge that is available to those with skill in the art. The issued U.S. patents, allowed applications, published foreign applications, and references cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

We Claim:

1. A method of treating amyotrophic lateral sclerosis (ALS) in a patient in need thereof comprising administering to the patient a therapeutically effective amount of a compound which inhibits SOD aggregation.

2. The method of claim 1, wherein the compound inhibits SOD aggregation by interacting with a region of SOD selected from the critical region or at least one beta barrel end.

3. The method of claim 2, wherein the compound blocks at least one amino acid in the critical region of SOD.

4. The method of claim 2, wherein the compound blocks Cys111 of SOD.

5. The method of claim 2, wherein the compound comprises one or more of the following:
(a) a chemical moiety comprising a functional group capable of interacting with the sulfhydryl side chain of a Cys-111 of SOD;
(b) a chemical moiety positioned to interact with the acidic side-chain of an Asp-109 of SOD;
(c) a chemical moiety positioned to interact with the imidazole of His 110 of SOD;
(d) a chemical moiety capable of specifically binding Cys-111;
(e) a chemical moiety capable of specifically binding Cys-111 and one or more amino acids within 5 amino acid residues of Cys-111;
(f) a chemical moiety capable of binding one or more amino acids within 5 amino acid residues of Cys-111;
(g) a chemical moiety capable of binding one or more amino acids located within 15 Å of Cys-111;
(h) a chemical moiety capable of binding one or more amino acids located within 10 Å of Cys-111;
(i) a chemical moiety capable of binding a basic amino acid within 15 angstroms of Cys-111;
(j) a chemical moiety capable of binding a basic amino acid located within 10 Å of Cys-111;
(k) a chemical moiety capable of binding a hydrophobic amino acid within 5 amino acid residues of Cys-111;
(l) a chemical moiety capable of binding a hydrophobic amino acid within 10 Å of Cys-111;
(m) a chemical moiety capable of binding an acidic amino acid within 5 amino acid residues of Cys-111; and
(n) a chemical moiety capable of binding an acidic amino acid located within 10 Å of Cys-111.

6. A method of treating ALS in a patient in need thereof comprising administering to the patient a therapeutically effective amount of a compound which inhibits SOD aggregation mediated by a Cys-111 of SOD.

7. The method of claim 6, wherein the compound inhibits SOD aggregation by interacting with the critical region of SOD.

8. The method of claim 6, wherein said compound binds Cys-111 or subsite thereof.

9. The method of claim 6, wherein the compound binds Cys-111 and one or more amino acids within 15 angstroms of Cys-111.

10. The method of claim 6, wherein the compound is 4-pyrimidinecarboxylic acid or orotic acid.

11. The method of claim 1, wherein the compound is orotic acid.

12. The method of claim 6, wherein the compound is orotic acid.

13. The method of claim 6, wherein the compound has a molecular weight of less than 2000 g/mol.

14. The method of claim 6, wherein the compound has a molecular weight of less than 1000 g/mol.

15. A method of identifying a compound which inhibits SOD aggregation comprising the steps of:
(a) identifying the amino acids in the critical region;
(b) rationally designing compounds which will react with one or more amino acids identified in (a); and
(c) screening the compounds identified in step (b) in an SOD aggregation assay.

16. A compound identified according to the method of claim 15.

17. A method of treating ALS in a patient in need thereof comprising administering to the patient a pharmaceutically acceptable compound of claim 16.

18. A method of designing a compound capable of inhibiting aggregation of SOD comprising the steps of (a) identifying one or more functional groups capable of interacting with one or more subsites of the critical region of SOD; and (b) identifying a scaffold which presents the functional group or functional groups identified in (a) in a suitable orientation for interacting with one or more subsites of the critical region of SOD.

19. The method of claim 18 further comprising the step of screening said compound in a SOD aggregation model.

20. A compound identified according to the method of claim 18.

21. The method of claim 2, wherein the compound interacts with at least one amino acid in the region of a SOD beta barrel end.

22. The method of claim 21 wherein a SOD beta barrel end is the S5-S6 beta barrel end.

23. The method of claim 22 wherein the compound interacts with any combination of amino acid residues 65-69, 74-81, 86-88, 95-99 or 102-103 of an S5-S6 beta barrel end.

24. The method of claim 21 wherein a SOD beta barrel end is the S 1-S8 beta barrel end.

25. The method of claim 24 wherein the compound interacts with any combination of amino acid residues 7-11 and 146-147.

26. A method of identifying a pharmaceutically acceptable compound which inhibits SOD aggregation mediated by a labile beta barrel end of SOD comprising the steps of:
(a) identifying the amino acids in the S5-S6 or S1-S8 clefts of a beta barrel end of SOD;
(b) rationally designing compounds which will react with one or more amino acids identified in (a); and
(c) screening the compounds identified in step (b) in an SOD aggregation assay.

27. A compound identified according to the method of claim 25.

28. A method of treating amyotrophic lateral sclerosis in a patient in need thereof comprising administering to the patient a pharmaceutically acceptable compound of claim 26.

29. A method of designing a compound capable of inhibiting aggregation of SOD comprising the steps of (a) identifying one or more functional groups capable of interacting with one or more subsites of the S5-S6 cleft or the S1-S8 cleft of a beta barrel end of SOD; and (b) identifying a scaffold which presents the functional group or functional groups identified in (a) in a suitable orientation for interacting with one or more subsites of the S5-S6 or S1-S8 clefts of a beta barrel end of SOD.

30. The method of claim 29 further comprising the step of screening said compound in a SOD aggregation model.

31. A compound identified according to the method of claim 29.

32. A method of treating a SOD aggregation-mediated disease in a patient in need thereof comprising administering to the patient a therapeutically effective amount of a compound which inhibits SOD aggregation by interacting with a region of SOD selected from the critical region or at least one beta barrel end.

33. The method of claim 32 wherein the SOD aggregation-mediated disease is a SOD aggregation-mediated motor neuron disease.

34. A method of treating a SOD aggregation-mediated disease in a patient in need thereof comprising administering to the patient a therapeutically effective amount of a compound represented by the Formula 3: 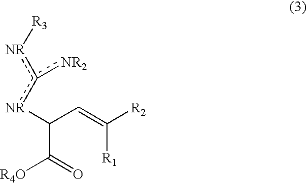
or pharmaceutically acceptable salts, esters, prodrugs, enantiomers, diastereoisomers, racemates, and tautomers thereof, wherein
R₁ is hydrogen, or a substituted or unsubstituted aliphatic or aromatic group;
R₂ is a substituted or unsubstituted aliphatic or aromatic group;
R₃ is a substituted or unsubstituted aliphatic or aromatic group;
R₄ is hydrogen, or an aliphatic or aromatic group;
each R at a guanidino nitrogen is independently absent or selected from a hydrogen, or a substituted or unsubstituted aliphatic or aromatic group, wherein at least one R is absent and one bond is a double bond; and
the dashed bond represents a single bond, a double bond or a tautomer.

35. The method of claim 34 wherein the SOD aggregation-mediated disease is a SOD aggregation-mediated motor neuron disease.

36. The method of claim 34 wherein the SOD aggregation-mediated disease is ALS.

37. The method of claim 34 wherein R₁ is a hydrophobic aliphatic group.

38. The method of claim 37 wherein R₁ is a C₃-C₉ alkyl group.

39. The method of claim 38 wherein the C₃-C₉ alkyl group is selected from a straight or branched chain butyl, pentyl, hexyl or octyl group.

40. The method of claim 34 wherein R₂ is a substituted or unsubstituted, saturated or unsaturated alkyl group.

41. The method of claim 40 wherein R₂ is a C₂-C₅ alkyl or alkenyl group.

42. The method of claim 41 wherein R₂ is a substituted or unsubstituted ethyl or propenyl group.

43. The method of claim 34 wherein R₃ is a substituted methylene group.

44. The method of claim 34 wherein one R is absent, one bond to a guanidino nitrogen is a double bond, and each remaining R is a hydrogen.

45. The method of claim 34 wherein the compound is a dimer of Formula 3.

46. The method of claim 45 wherein the dimer occurs at R₃ wherein the R₃ group has the structure L-R₈ where R8 has the Formula 3, and L is a ligand.

47. The method of claim 46 wherein L is a C₂-C₈ substituted or unsubstituted alkylene.

48. The method of claim 34 wherein the compound has the Formula 4: 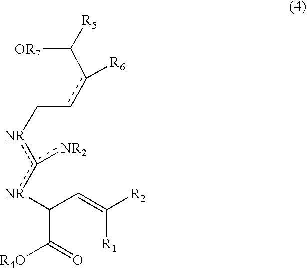
wherein R, R1, R2, and R4 are as defined in claim 34;
R₅ is H, carboxylic acid, sulfonic acid, sulfate and esters thereof, amino, amido, imino, hydroxy or an aliphatic or aromatic group;
R₆ is H, carboxy, sulfonic acid, sulfate and esters thereof, amino, amido, urea, acylurea, ureacarbonyl hydroxy, nitroso, nitro, or a substituted or unsubstituted aliphatic or aromatic group;
or R₅ and R₆ can be taken together to form a ring, including substituted or unsubstituted polycyclic ring systems;
R₇ is a hydrogen, substituted or unsubstituted alkyl, acyl group, or protecting group; and
the dashed bond represents a single bond, a double bond or a tautomer.

49. The method of claim 48 wherein R₅ is a substituted or unsubstituted straight or branched chain, saturated or unsaturated C₁-C₈ alkyl.

50. The method of claim 49 wherein R₅ is a substituted or unsubstituted straight or branched chain, saturated or unsaturated ethyl or pentyl.

51. The method of claim 48 wherein the compound is selected from:

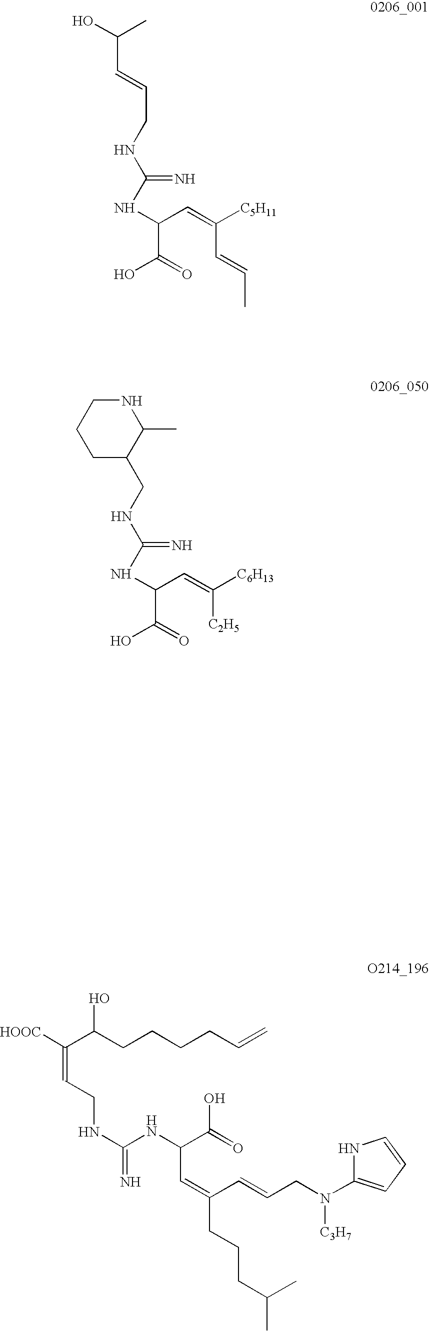

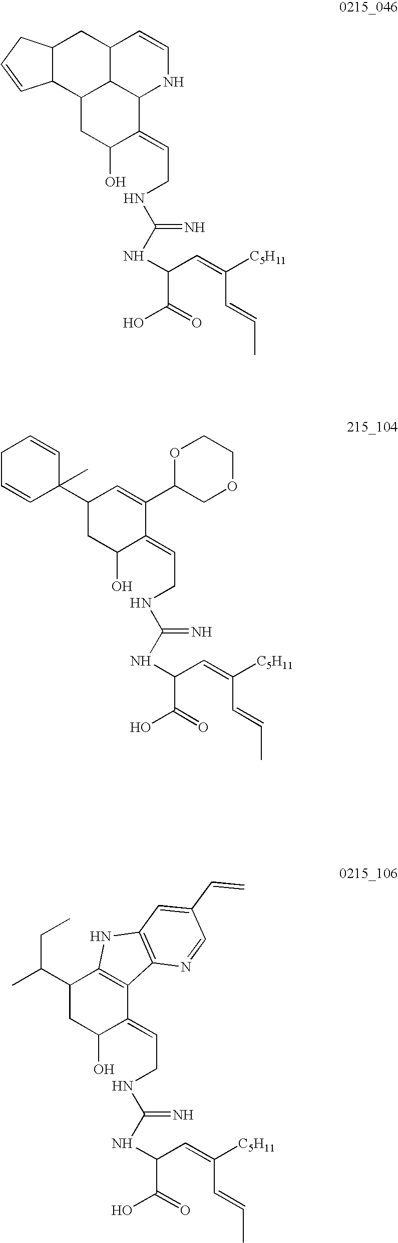

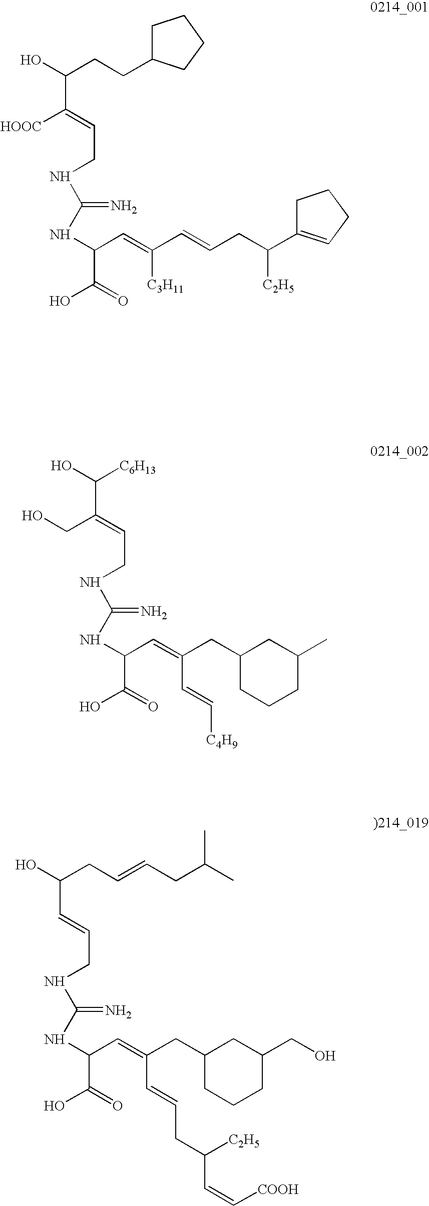

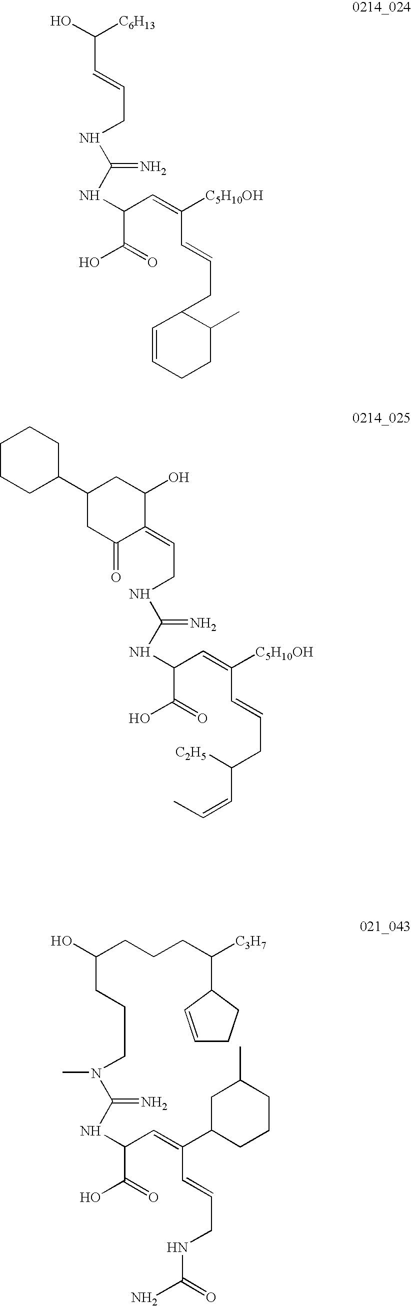

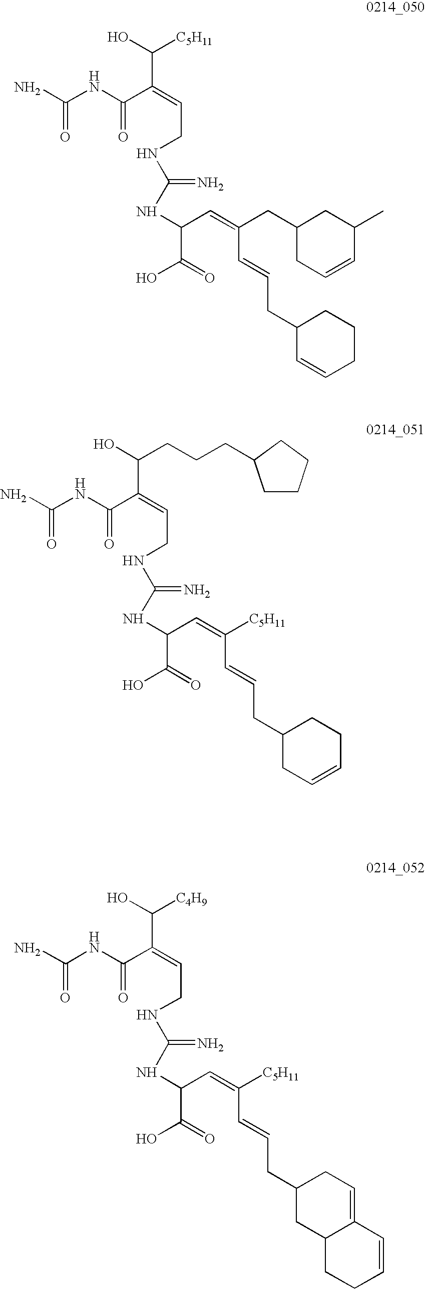

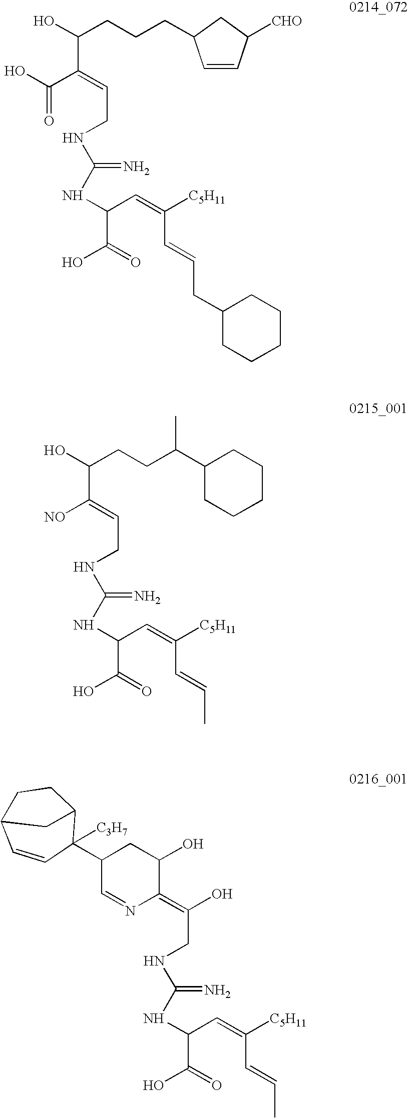

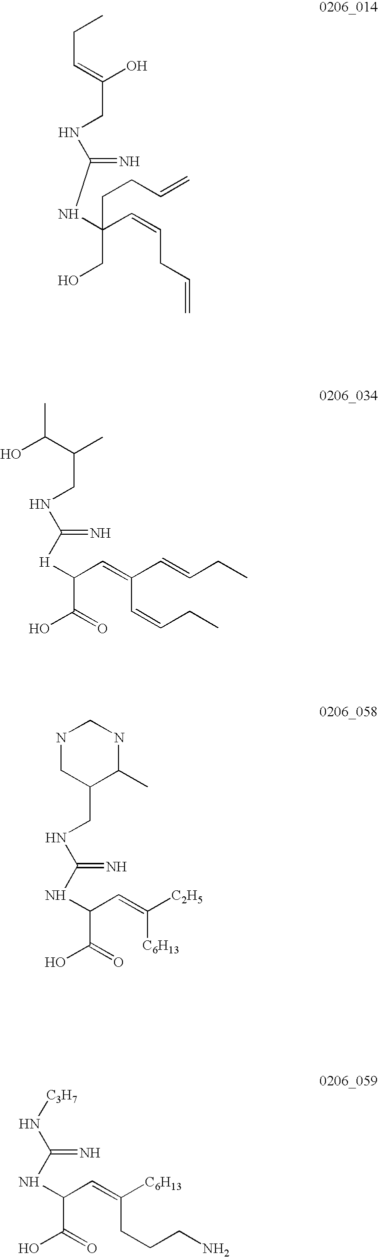

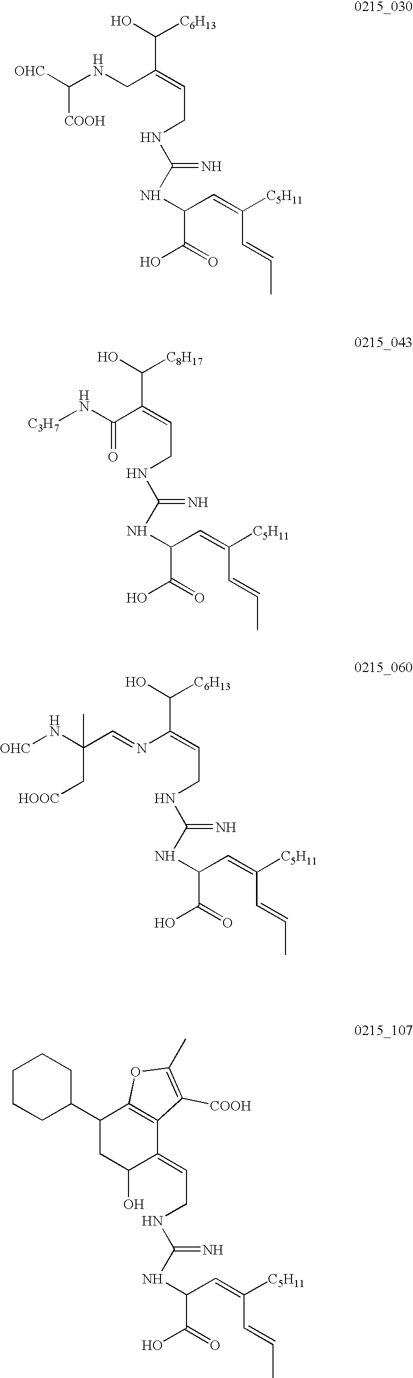

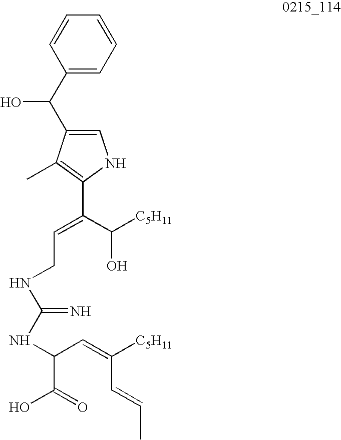

52. A compound represented by the Formula 4: 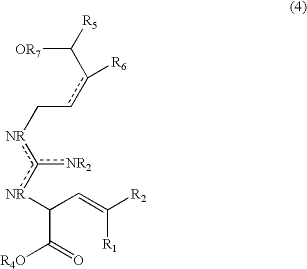
wherein R, R1, R2, and R4 are as defined in claim 34;
R₅ is H, carboxylic acid, sulfonic acid, sulfate and esters thereof, amino, amido, imino, hydroxy or an aliphatic or aromatic group;
R₆ is H, carboxy, sulfonic acid, sulfate and esters thereof, amino, amido, urea, acylurea, ureacarbonyl hydroxy, nitroso, nitro, or a substituted or unsubstituted aliphatic or aromatic group;
or R₅ and R₆ can be taken together to form a ring, including substituted or unsubstituted polycyclic ring systems;
R₇ is a hydrogen, substituted or unsubstituted alkyl, acyl group, or protecting group; and
the dashed bond represents a single bond, a double bond or a tautomer.


SEQUENCE LISTING (1 sequence)

SEQ ID NO: 1 - protein, 153 aa, Homo sapiens
ATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ